SEQUENCE LISTING (8 sequences)

SEQ ID NO: 1 - DNA, 1557 bp, HOMO SAPIENS
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggccatatgcaccatcatcatcatcattcttctggtctggtgccacgcggttctggtatgaaagaaaccgctgctgctaaattcgaacgccagcacatggacagcccagatctgggtaccgacgacgacgacaaggccatggctgatatcggatccgaattccgagcgctgcgcgaggcgctctgccctgagccctgcaactgcgtgcccgacggcgccctgcgctgccccggccccacggccggtctcactcgactatcacttgcctacctccctgtcaaagtgatcccatctcaagctttcagaggacttaatgaggtcataaaaattgaaatctctcagattgattccctggaaaggatagaagctaatgcctttgacaacctcctcaatttgtctgaaatactgatccagaacaccaaaaatctgagatacattgagcccggagcatttataaatcttccccgattaaaatacttgagcatctgtaacacaggcatcagaaagtttccagatgttacgaaggtcttctcctctgaatcaaatttcattctggaaatttgtgataacttacacataaccaccataccaggaaatgcttttcaagggatgaataatgaatctgtaacactcaaactatatggaaatggatttgaagaagtacaaagtcatgcattcaatgggacgacactgacttcactggagctaaaggaaaacgtacatctggagaagatgcacaatggagccttccgtggggccacagggccgaaaaccttggatatttcttccaccaaattgcaggccctgccgagctatggcctagagtccattcagaggctaattgccacgtcatcctattctctaaaaaaattgccatcaagagaaacatttgtcaatctcctggaggccacgttgacttaccccagccactgctgtgcttttagaaacttgccaacaaaagaacagaatttttcacattccatttctgaaaacttttccaaacaatgtgaaagcacagtaaggaaagtgaataacaaaacactttattcttccatgcttgctgagagtgaactgagtggctgggactatgaatatggtttctgcttacccaagacaccccgatgtgctcctgaaccagatgcttttaatccctgtgaagatattatgggcgtcgacaagcttgcggccgcactcgagcaccaccaccaccaccactga

SEQ ID NO: 2 - protein, 518 aa, Homo Sapiens
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDKAMADIGSEFRALREALCPEPCNCVPDGALRCPGPTAGLTRLSLAYLPVKVIPSQAFRGLNEVIKIEISQIDSLERIEANAFDNLLNLSEILIQNTKNLRYIEPGAFINLPRLKYLSICNTGIRKFPDVTKVFSSESNFILEICDNLHITTIPGNAFQGMNNESVTLKLYGNGFEEVQSHAFNGTTLTSLELKENVHLEKMHNGAFRGATGPKTLDISSTKLQALPSYGLESIQRLIATSSYSLKKLPSRETFVNLLEATLTYPSHCCAFRNLPTKEQNFSHSISENFSKQCESTVRKVNNKTLYSSMLAESELSGWDYEYGFCLPKTPRCAPEPDAFNPCEDIMGVDKLAAALEHHHHHH

SEQ ID NO: 3 - DNA, 1551 bp, Homo Sapiens
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggccatatgcaccatcatcatcatcattcttctggtctggtgccacgcggttctggtatgaaagaaaccgctgctgctaaattcgaacgccagcacatggacagcccagatctgggtaccgacgacgacgacaaggccatggctgatatcggatccgaattcagggtttttctctgccaagagagcaaggtgacagagattccttctgacctcccgaggaatgccattgaactgaggtttgtcctcaccaagcttcgagtcatccaaaaaggtgcattttcaggatttggggacctggagaaaatagagatctctcagaatgatgtcttggaggtgatagaggcagatgtgttctccaaccttcccaaattacatgaaattagaattgaaaaggccaacaacctgctctacatcacccctgaggccttccagaaccttcccaaccttcaatatctgttaatatccaacacaggtattaagcaccttccagatgttcacaagattcattctctccaaaaggttttacttgacattcaagataacataaacatccacacaattgaaagaaattctttcgtggggctgagctttgaaagtgtgattctatggctgaataagaatgggattcaagaaatacacaactgtgcattcaatggaacccaactagatgcagtgaatctaagcgataataataatttagaagaattgcctaatgatgttttccacggagcctctggaccagtcattctagatatttcaagaacaaggatccattccctgcctagctatggcttagaaaatcttaagaagctgagggccaggtcgacttacaacttaaaaaagctgcctactctggaaaagcttgtcgccctcatggaagccagcctcacctatcccagccattgctgtgcctttgcaaactggagacggcaaatctctgagcttcatccaatttgcaacaaatctattttaaggcaagaagttgattatatgactcaggctaggggtcagagatcctctctggcagaagacaatgagtccagctacagcagaggatttgacatgacgtacactgagtttgactatgacttatgcaatgaagtggttgacgtgacctgctcccctaagccagatgcattcaacccatgtgaagatatcatgggggtcgacaagcttgcggccgcactcgagcaccaccaccaccaccactga

SEQ ID NO: 4 - protein, 516 aa, Homo Sapiens
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDKAMADIGSEFRVFLCQESKVTEIPSDLPRNAIELRFVLTKLRVIQKGAFSGFGDLEKIEISQNDVLEVIEADVFSNLPKLHEIRIEKANNLLYITPEAFQNLPNLQYLLISNTGIKHLPDVHKIHSLQKVLLDIQDNINIHTIERNSFVGLSFESVILWLNKNGIQEIHNCAFNGTQLDAVNLSDNNNLEELPNDVFHGASGPVILDISRTRIHSLPSYGLENLKKLRARSTYNLKKLPTLEKLVALMEASLTYPSHCCAFANWRRQISELHPICNKSILRQEVDYMTQARGQRSSLAEDNESSYSRGFDMTYTEFDYDLCNEVVDVTCSPKPDAFNPCEDIMGVDKLAAALEHHHHHH

SEQ ID NO: 5 - DNA, 2100 bp, Homo Sapiens
atgaagcagcggttctcggcgctgcagctgctgaagctgctgctgctgctgcagccgccgctgccacgagcgctgcgcgaggcgctctgccctgagccctgcaactgcgtgcccgacggcgccctgcgctgccccggccccacggccggtctcactcgactatcacttgcctacctccctgtcaaagtgatcccatctcaagctttcagaggacttaatgaggtcataaaaattgaaatctctcagattgattccctggaaaggatagaagctaatgcctttgacaacctcctcaatttgtctgaaatactgatccagaacaccaaaaatctgagatacattgagcccggagcatttataaatcttccccgattaaaatacttgagcatctgtaacacaggcatcagaaagtttccagatgttacgaaggtcttctcctctgaatcaaatttcattctggaaatttgtgataacttacacataaccaccataccaggaaatgcttttcaagggatgaataatgaatctgtaacactcaaactatatggaaatggatttgaagaagtacaaagtcatgcattcaatgggacgacactgacttcactggagctaaaggaaaacgtacatctggagaagatgcacaatggagccttccgtggggccacagggccgaaaaccttggatatttcttccaccaaattgcaggccctgccgagctatggcctagagtccattcagaggctaattgccacgtcatcctattctctaaaaaaattgccatcaagagaaacatttgtcaatctcctggaggccacgttgacttaccccagccactgctgtgcttttagaaacttgccaacaaaagaacagaatttttcacattccatttctgaaaacttttccaaacaatgtgaaagcacagtaaggaaagtgaataacaaaacactttattcttccatgcttgctgagagtgaactgagtggctgggactatgaatatggtttctgcttacccaagacaccccgatgtgctcctgaaccagatgcttttaatccctgtgaagatattatgggctatgacttccttagggtcctgatttggctgattaatattctagccatcatgggaaacatgactgttctttttgttctcctgacaagtcgttacaaacttacagtgcctcgttttctcatgtgcaatctctcctttgcagacttttgcatggggctctatctgctgctcatagcctcagttgattcccaaaccaagggccagtactataaccatgccatagactggcagacagggagtgggtgcagcactgctggctttttcactgtattcgcaagtgaactttctgtctacaccctcaccgtcatcactctagaaagatggcacaccatcacctatgctattcacctggaccaaaagctgcgattaagacatgccattctgattatgcttggaggatggctcttttcttctctaattgctatgttgccccttgtcggtgtcagcaattacatgaaggtcagtatttgcttccccatggatgtggaaaccactctctcacaagtctatatattaaccatcctgattctcaatgtggtggccttcttcataatttgtgcttgctacattaaaatttattttgcagttcgaaacccagaattaatggctaccaataaagatacaaagattgctaagaaaatggcaatcctcatcttcaccgatttcacctgcatggcacctatctctttttttgccatctcagctgccttcaaagtacctcttatcacagtaaccaactctaaagttttactggttcttttttatcccatcaattcttgtgccaatccatttctgtatgcaatattcactaagacattccaaagagatttctttcttttgctgagcaaatttggctgctgtaaacgtcgggctgaactttatagaaggaaagatttttcagcttacacctccaactgcaaaaatggcttcactggatcaaataagccttctcaatccaccttgaagttgtccacattgcactgtcaaggtacagctctcctagacaagactcgctacacagagtgttaa

SEQ ID NO: 6 - protein, 699 aa, Homo Sapiens
MKQRFSALQLLKLLLLLQPPLPRALREALCPEPCNCVPDGALRCPGPTAGLTRLSLAYLPVKVIPSQAFRGLNEVIKIEISQIDSLERIEANAFDNLLNLSEILIQNTKNLRYIEPGAFINLPRLKYLSICNTGIRKFPDVTKVFSSESNFILEICDNLHITTIPGNAFQGMNNESVTLKLYGNGFEEVQSHAFNGTTLTSLELKENVHLEKMHNGAFRGATGPKTLDISSTKLQALPSYGLESIQRLIATSSYSLKKLPSRETFVNLLEATLTYPSHCCAFRNLPTKEQNFSHSISENFSKQCESTVRKVNNKTLYSSMLAESELSGWDYEYGFCLPKTPRCAPEPDAFNPCEDIMGYDFLRVLIWLINILAIMGNMTVLFVLLTSRYKLTVPRFLMCNLSFADFCMGLYLLLIASVDSQTKGQYYNHAIDWQTGSGCSTAGFFTVFASELSVYTLTVITLERWHTITYAIHLDQKLRLRHAILIMLGGWLFSSLIAMLPLVGVSNYMKVSICFPMDVETTLSQVYILTILILNVVAFFIICACYIKIYFAVRNPELMATNKDTKIAKKMAILIFTDFTCMAPISFFAISAAFKVPLITVTNSKVLLVLFYPINSCANPFLYAIFTKTFQRDFFLLLSKFGCCKRRAELYRRKDFSAYTSNCKNGFTGSNKPSQSTLKLSTLHCQGTALLDKTRYTEC

SEQ ID NO: 7 - DNA, 2088 bp, Homo Sapiens
atggccctgctcctggtctctttgctggcattcctgagcttgggctcaggatgtcatcatcggatctgtcactgctctaacagggtttttctctgccaagagagcaaggtgacagagattccttctgacctcccgaggaatgccattgaactgaggtttgtcctcaccaagcttcgagtcatccaaaaaggtgcattttcaggatttggggacctggagaaaatagagatctctcagaatgatgtcttggaggtgatagaggcagatgtgttctccaaccttcccaaattacatgaaattagaattgaaaaggccaacaacctgctctacatcacccctgaggccttccagaaccttcccaaccttcaatatctgttaatatccaacacaggtattaagcaccttccagatgttcacaagattcattctctccaaaaggttttacttgacattcaagataacataaacatccacacaattgaaagaaattctttcgtggggctgagctttgaaagtgtgattctatggctgaataagaatgggattcaagaaatacacaactgtgcattcaatggaacccaactagatgcagtgaatctaagcgataataataatttagaagaattgcctaatgatgttttccacggagcctctggaccagtcattctagatatttcaagaacaaggatccattccctgcctagctatggcttagaaaatcttaagaagctgagggccaggtcgacttacaacttaaaaaagctgcctactctggaaaagcttgtcgccctcatggaagccagcctcacctatcccagccattgctgtgcctttgcaaactggagacggcaaatctctgagcttcatccaatttgcaacaaatctattttaaggcaagaagttgattatatgactcaggctaggggtcagagatcctctctggcagaagacaatgagtccagctacagcagaggatttgacatgacgtacactgagtttgactatgacttatgcaatgaagtggttgacgtgacctgctcccctaagccagatgcattcaacccatgtgaagatatcatggggtacaacatcctcagagtcctgatatggtttatcagcatcctggccatcactgggaacatcatagtgctagtgatcctaactaccagccaatataaactcacagtccccaggttccttatgtgcaacctggcctttgctgatctctgcattggaatctacctgctgctcattgcatcagttgatatccataccaagagccaatatcacaactatgccattgactggcaaactggggcaggctgtgatgctgctggctttttcactgtctttgccagtgagctgtcagtctacactctgacagctatcaccttggaaagatggcataccatcacgcatgccatgcagctggactgcaaggtgcagctccgccatgctgccagtgtcatggtgatgggctggatttttgcttttgcagctgccctctttcccatctttggcatcagcagctacatgaaggtgagcatctgcctgcccatggatattgacagccctttgtcacagctgtatgtcatgtccctccttgtgctcaatgtcctggcctttgtggtcatctgtggctgctatatccacatctacctcacagtgcggaaccccaacatcgtgtcctcctctagtgacaccaggatcgccaagcgcatggccatgctcatcttcactgacttcctctgcatggcacccatttctttctttgccatttctgcctccctcaaggtgcccctcatcactgtgtccaaagcaaagattctgctggttctgtttcaccccatcaactcctgtgccaaccccttcctctatgccatctttaccaaaaactttcgcagagatttcttcattctgctgagcaagtgtggctgctatgaaatgcaagcccaaatttataggacagaaacttcatccactgtccacaacacccatccaaggaatggccactgctcttcagctcccagagtcaccagtggttccacttacatacttgtccctctaagtcatttagcccaaaactaa

SEQ ID NO: 8 - protein, 695 aa, Homo Sapiens [sequence omitted]

(19) United States
(12) Patent Application Publication
(10) Pub. No.: US 2002/0128190 A1
(43) Pub. Date: Sep. 12, 2002

(54) Expression of properly folded and soluble extracellular domain of a gonadotropin receptor

(76) Inventors: Leslie Lobel, Riverdale, NY (US); Joyce Lustbader, Tenafly, NJ (US)

Correspondence Address: John P. White, Cooper & Dunham LLP, 1185 Avenue of the Americas, New York, NY, 10036, US

(21) Appl. No.: 09/804,626

(22) Filed: Mar. 9, 2001

(51) Int. Cl. A61K 38/00; C12P 21/02; C12N 5/06; C07H 21/04; C07K 14/705

(52) U.S. Cl. 514/12; 530/350; 536/23.5; 435/69.1; 435/320.1; 435/325

(57) ABSTRACT

This invention provides a nucleic acid which encodes a soluble polypeptide which comprises the extracellular domain of a gonadotropin receptor and thioredoxin, wherein the soluble polypeptide is capable of binding to the gonadotropin. This invention also provides the polypeptides encoded by these nucleic acids and methods to produce the polypeptides. This invention also provides methods of preventing or terminating pregnancy, preventing or treating cancer, and decreasing androgen and estrogen production.

The invention described herein was made with Government support under grant number DK-51266 from the National Institutes of Health. Accordingly, the United States Government has certain rights in this invention.

Throughout this application, various publications are referenced within parentheses. Disclosures of these publications in their entireties are hereby incorporated by reference into this application to more fully describe the state of the art to which this invention pertains. Full bibliographic citations for these references may be found immediately preceding the claims.

### BACKGROUND OF THE INVENTION

The expression of mammalian proteins in E. coli has provided an economical and facile method for producing large quantities of recombinant proteins (1,2). Proteins produced in this way can be useful for biochemical studies such as structural determination as well as for assay development or the identification of pharmacologically useful compounds with high throughput screening techniques (3,4). In addition, recombinant proteins produced in non-mammalian systems can potentially be useful as therapeutic agents (2).

The structural and functional aspects of the human luteinizing/choriogonadotropin (hLH/CG) receptor are being studied. The extracellular domain of the receptor has been expressed as a fusion with the capsid protein III (cpIII) of filamentous phage (5). The fusion phage bound hCG specifically and with comparable affinity to that of native receptor and in the proper orientation (5). Although the cpIII phage display system has proven to be useful in high throughput screening protocols for antagonists of the hLH/CG receptor and other proteins (L. I. Lobel, J. P. Horseman, X. Zeng, J. W. Lustbader, H. Chen, F. C. T. Allnutt, submitted) it is not a useful expression technique for production of large quantities of a protein. The incorporation of a single copy of the recombinant protein on the surface of the phage, on average, and limitations on the titer of phage particles greatly reduces the utility of this method as an expression technique. Alternative phage display systems such as the cpVIII gene of filamentous phage offers increased expression of the desired fusion protein since there are approximately 2700 copies of the gene product on the surface of each filamentous phage (6,7). Nonetheless, as the size of the fusion protein increases incorporation in the capsid of the fusion product decreases. Although the extracellular domain of the hLH/CG receptor can be fused to the cpVIII gene and folded properly on the surface of the phage, less than fifty copies, on average, are incorporated in the capsid of a single phage (L. Lobel, unpublished observations). These fusion phage are therefore incapable of expressing the receptor binding domain at a level that would be feasible for producing large quantities of this protein.

Earlier attempts at expressing the extracellular domain of the hLH/CG receptor employed eukaryotic, prokaryotic and insect cell vectors. Although baculovirus based systems can in theory express sufficient quantities of material, our previous attempts at expression in this system failed to produce large enough quantities of material that could bind hCG with a high affinity.

Whereas Bahl has previously reported the production of the hormone binding domain in E. coli, this material was not folded correctly and had to be refolded in vitro (8). This procedure is laborious and generally not useful for the isolation of large quantities of homogenous proteins. Successful isolation of the extracellular domain of the hLH/CG receptor from a chimerically expressed eukaryotic protein has been previously reported (9). Nonetheless, this method of expression is relatively laborious and costly and cannot provide sufficient material for detailed structural studies of the binding domain of the receptor.

To obviate the problems and limitations previously encountered with other expression techniques the expression of the extracellular domain of the receptor in an E. coli system was re-examined. Studies on protein expression in E. coli have demonstrated that the environment in bacterial cytoplasm does not favor disulfide bond formation in cytoplasmically expressed recombinant proteins. Nonetheless, the thioredoxin reductase (trxB) and glutathione reductase (gor) genes play a major role in the thiol-disulfide balance in the E. coli cytoplasm (10,11). Mutants of E. coli in either or both of these genes tend to favor disulfide bond formation in cytoplasmically expressed recombinant proteins (10). Therefore the expression of the extracellular domain of the hLH/CG receptor in an E. coli strain containing mutations in these reductase pathways was pursued.

These studies demonstrate that the hLH/CG or the hFSH extracellular domains have been successfully expressed in the cytoplasm of E.coli as inducible fusion proteins with thioredoxin in both a trxB host and a trxb/gor host strain that is mutant in both reductases. The recombinant soluble proteins are folded properly and can bind their respective hormone ligands specifically, with affinities close to the native intact receptors. Furthermore, following a three hour induction, 10-30 mg per liter of fusion protein can be obtained in this expression system. The production of large quantities of the extracellular domain of the hLH/CG or hFSH receptors that are folded properly will be useful for the generation of a panel of epitope specific anti-receptor monoclonal antibodies and structural analysis of the hLH/CG or hFSH receptors by X-ray crystallography. Furthermore, large quantities of a soluble form of the hLH/CG or hFSH receptors might be useful clinically to regulate the circulating levels of hormones for contraception or treatment of a variety of neoplasias that appear to respond in particular to hCG in either an autocrine or paracrine fashion (12).

### SUMMARY OF THE INVENTION

This invention provides a nucleic acid which encodes a soluble polypeptide which comprises an extracellular domain of a gonadotropin receptor and thioredoxin, wherein the soluble polypeptide is capable of binding to the gonadotropin. This invention provides a nucleic acid which encodes a soluble polypeptide which comprises an extracellular domain of a gonadotropin receptor and a peptide segment comprising consecutive histidine residues, wherein the soluble polypeptide is capable of binding to the gonadotropin. This invention also provides a nucleic acid which encodes a soluble polypeptide which comprises an extracellular domain of a gonadotropin receptor and thioredoxin and further comprises a peptide segment comprising consecutive histidine residues.

In one embodiment of the above nucleic acids, the gonadotropin receptor is a human luteinizing hormone/choriogonadotropin receptor and the soluble polypeptide is capable of binding to human luteinizing hormone or human chorionic gonadotropin.

In one embodiment of the above nucleic acids, the gonadotropin receptor is a human follicle stimulating hormone receptor and the soluble polypeptide is capable of binding to human follicle stimulating hormone.

This invention provides a soluble polypeptide which comprises an extracellular domain of a gonadotropin receptor and thioredoxin, wherein the soluble polypeptide is capable of binding to the gonadotropin.

This invention provides a soluble polypeptide which comprises an extracellular domain of a gonadotropin receptor and a peptide segment comprising consecutive histidine residues, wherein the soluble polypeptide is capable of binding to the gonadotropin.

This invention provides a soluble polypeptide which comprises an extracellular domain of a gonadotropin receptor and thioredoxin and a peptide segment comprising consecutive histidine residues, wherein the soluble polypeptide is capable of binding to the gonadotropin.

In one embodiment of the soluble polypeptide, the gonadotropin receptor is a human luteinizing hormone/choriogonadotropin receptor and the soluble polypeptide is capable of binding to human luteinizing hormone or human chorionic gonadotropin.

In one embodiment of the soluble polypeptide, the gonadotropin receptor is a human follicle stimulating hormone receptor and the soluble polypeptide is capable of binding to human follicle stimulating hormone.

This invention provides a method of identifying an antibody capable of binding to an extracellular domain of a gonadotropin receptor which comprises:

(a) administering a polypeptide of the subject invention to a subject and obtaining antiserum from the subject;

(b) contacting a gonadotropin receptor with the antiserum;

(c) determining whether any antibody present in the antiserum binds to the a gonadotropin receptor and isolating such antibody, so as to thereby identify an antibody capable of binding to the extracellular domain of a gonadotropin receptor.

This invention provides a method of obtaining a composition which comprises:

(a) identifying an antibody capable of binding to an extracellular domain of a gonadotropin receptor by the above method; and

(b) admixing the antibody so identified with a carrier.

This invention provides a method of preventing a subject from becoming pregnant which comprises administering to the subject an amount of an antibody effective to bind to an extracellular domain of a gonadotropin receptor so as to thereby prevent the subject from becoming pregnant.

This invention provides a method of preventing a subject from becoming pregnant which comprises administering to the subject an amount of a polypeptide of the subject invention or an extracellular domain of a gonadotropin receptor so as to thereby prevent a subject from becoming pregnant.

This invention provides a method of terminating a pregnancy in a subject which comprises administering to the subject an amount of an antibody effective to bind to an extracellular domain of a gonadotropin receptor so as to thereby terminate the pregnancy in the subject. This invention provides a method of terminating a pregnancy in a subject which comprises administering to the subject an amount of a polypeptide of the subject invention or an extracellular domain of a gonadotropin receptor so as to thereby terminate the pregnancy in the subject.

This invention provides a method of stimulating or enhancing production of an antibody capable of binding to an extracellular domain of a gonadotropin receptor in a subject which comprises administering to the subject an amount of a polypeptide of the subject invention or an extracellular domain of a gonadotropin receptor effective to stimulate or enhance antibody production in the subject.

This invention provides a method of treating a cancer in a subject which comprises administering to the subject an amount of a polypeptide of the subject invention or an extracellular domain of a gonadotropin receptor effective to stimulate or enhance production of an antibody capable of binding to an extracellular domain of a gonadotropin receptor so as to thereby treat the cancer in the subject.

This invention provides a method of treating a cancer in a subject which comprises administering to the subject an amount of an antibody effective to bind to an extracellular domain of a gonadotropin receptor so as to thereby treat a cancer in the subject.

This invention provides a method of preventing a cancer in a subject which comprises administering to the subject an amount of a polypeptide of the subject invention or an extracellular domain of a gonadotropin receptor effective to stimulate or enhance production of an antibody capable of binding to an extracellular domain of a gonadotropin receptor so as to thereby prevent the cancer in the subject.

This invention provides a method of preventing a cancer in a subject which comprises administering to the subject an amount of an antibody effective to bind to an extracellular domain of a gonadotropin receptor so as to thereby prevent a cancer in the subject.

This invention provides a method of decreasing a subject's production of androgen which comprises administering to the subject an amount of a polypeptide of the subject invention or an extracellular domain of a gonadotropin receptor effective to stimulate production of an antibody capable of binding to an extracellular domain of a gonadotropin receptor in the subject, so as to thereby decrease the subject's production of androgen.

This invention provides a method of decreasing a subject's production of androgen which comprises administering to the subject an amount of an antibody effective to bind to the extracellular domain of a gonadotropin receptor so as to thereby decrease the subject's production of androgen.

This invention provides a method of preventing a subject from becoming afflicted with ovarian hyperstimulatory syndrome which comprises administering to the subject an amount of an antibody effective to bind to an extracellular domain of a gonadotropin receptor so as to thereby prevent the subject from becoming afflicted with ovarian hyperstimulatory syndrome.

This invention provides a method of treating a subject afflicted with ovarian hyperstimulatory syndrome which comprises administering to the subject an amount of an antibody effective to bind to an extracellular domain of a gonadotropin receptor so as to thereby treat the subject afflicted with ovarian hyperstimulatory syndrome.

### BRIEF DESCRIPTION OF THE FIGURES

FIG. 1: Diagram of LR4 vector:

This figure illustrates the construction of the LR4 vector from pET32a(+) and sequences encoding the extracellular domain of the hLH/CG receptor.

FIG. 2: Protein gels and Western blots:

Panel A contains lanes from non-reducing SDS polyacrylamide gels. Lane C contains a protein extract from the host strain (Origami DE3 pLysS) induced with IPTG for 3 hours. This lane serves as a control. An extract from the same strain containing the LR4 vector and induced for 1.5 hours with IPTG is in lane 1 (extracts from bacteria following a 3 hour induction overloads the lane of the gel). Lane 2 contains a sample of the washed and resolubilized protein aggregate. Lane 3 contains a sample of the purified protein following Ni-NTA chromatography. Lanes C and 1 were cut from the same polyacrylamide gel whereas lanes 2 and 3 were from different protein gels. The major protein band migrating at 57 Kd corresponds to the size of the thioredoxin-receptor fusion protein. All samples were boiled before loading to avoid protein aggregation that occurs in the SDS gel buffer.

Panel B contains Western blot analysis of the purified fusion protein that was probed with monoclonal anti-hLH/CG receptor antibodies and also polyclonal anti-hLH/CG receptor antisera. Note that a single major species (see arrows) is identified in both blots. Samples for the Western blots were not boiled before loading since this reduced affinity of the antibodies to the fusion protein. As a result there is some aggregated protein at the top of the gel.

FIG. 3: Affinity of fusion protein for hCG:

This affinity curve for the receptor-thioredoxin fusion protein was generated according to the techniques outlined in the methods section. Fixed amounts of fusion protein and cpm of labeled hCG were incubated in the presence of varying concentrations of unlabeled hCG. Bound labeled hCG was isolated from the incubation mixture with Ni-NTA resin and counted. Molarity of unlabeled hCG in each incubation reaction was graphed versus the percent of maximally bound counts as defined in material and methods. HFSH was also tested in this assay to demonstrate the specificity of the fusion protein for hCG. The Standard error for all points is very small and as a result error bars are omitted for clarity. Each graph point represents the mean of five binding experiments.

FIG. 4: Effect of anti-hCG monoclonal antibodies on hCG binding to fusion protein.

To determine that hCG binds the receptor-thioredoxin fusion protein in the correct orientation we tested the effect of anti-hCG B105 and B107 on binding of labeled hCG to fusion protein. B105 can bind hCG while bound to the native receptor and does not interfere with binding of labeled hCG to fusion protein. B107, on the other hand, blocks binding of hCG to native receptor and likewise blocks binding of labeled hCG to the fusion protein. We conclude, therefore, that hCG is bound by the fusion protein in the proper orientation.

FIG. 5: Diagram of FR8 vector:

This figure illustrates the construction of the FR8 vector from pET32a(+) and sequences encoding the extracellular domain of the hFSH receptor.

FIG. 6

Nucleotide (Panel A) (SEQ ID NO:1) and translated amino acid sequence (Panel B) (SEQ ID NO:2) for the Pet32a(+) hLH/CG Receptor Sequence.

FIG. 7

Nucleotide (Panel A) (SEQ ID NO:3) and translated amino acid sequence (Panel B) (SEQ ID NO:4) for the Pet32a(+) hFSH Receptor Sequence.

### DETAILED DESCRIPTION OF THE INVENTION

This invention provides a nucleic acid which encodes a soluble polypeptide which comprises an extracellular domain of a gonadotropin receptor and thioredoxin, wherein the soluble polypeptide is capable of binding to the gonadotropin. This invention provides a nucleic acid which encodes a soluble polypeptide which comprises an extracellular domain of a gonadotropin receptor and a peptide segment comprising consecutive histidine residues, wherein the soluble polypeptide is capable of binding to the gonadotropin. This invention also provides a nucleic acid which encodes a soluble polypeptide which comprises an extracellular domain of a gonadotropin receptor and thioredoxin and further comprises a peptide segment comprising consecutive histidine residues.

In one embodiment of the above nucleic acids, the gonadotropin receptor is a human luteinizing hormone/choriogonadotropin receptor and the soluble polypeptide is capable of binding to human luteinizing hormone or human chorionic gonadotropin. In one embodiment, the extracellular domain of human luteinizing hormone/choriogonadotropin receptor comprises consecutive amino acids having the sequence set forth in SEQ ID NO:2 from the R or arginine at position 168 to the G or glycine at position 509.

In one embodiment, the soluble polypeptide comprises the entire extracellular domain of the receptor. In one embodiment, the soluble polypeptide comprises a portion or fragment of the extracellular domain of the receptor.

In one embodiment, the thioredoxin comprises consecutive amino acids having the sequence set forth in SEQ ID NO:2 from the M or methionine at position 1 to the A or alanine at position 109. In one embodiment, the cDNA sequence which encodes the extracellular domain comprises consecutive nucleotides having the sequence set forth in SEQ ID NO:1 from the C or cytosine at position 502 to the C or cytosine at position 1509. In one embodiment, the cDNA sequence which encodes thioredoxin is set forth in SEQ ID NO:l from the A or adenosine at position 1 to the C or cytosine at position 327.

The cDNA sequence for the full length human luteinizing hormone/choriogonadotropin receptor is set forth in SEQ ID NO:5. The amino acid sequence for the full length human luteinizing hormone/choriogonadotropin receptor is set forth in SEQ ID NO:6.

As used herein, “human luteinizing hormone/choriogonadotropin receptor” may be abbreviated hLH/CG receptor. This receptor may bind two hormones: human luteinizing hormone (“hLH”); or human chorionic gonadotropin (“hCG”).

In one embodiment of the above nucleic acids, the gonadotropin receptor is a human follicle stimulating hormone receptor and the soluble polypeptide is capable of binding to human follicle stimulating hormone. In one embodiment, the extracellular domain comprises consecutive amino acids having the sequence set forth in SEQ ID NO:4 from the R or arginine at position 168 to the G or glycine at position 501.

In one embodiment, the soluble polypeptide comprises the entire extracellular domain of the receptor. In one embodiment, the soluble polypeptide comprises a portion or fragment of the extracellular domain of the receptor.

For example, in one embodiment, the portion of the extracellular domain lacks the first 10 amino acids of the amino terminal portion of the extracellular domain. Accordingly, in one embodiment, the extracellular domain comprises consecutive amino acids having the sequence set forth in SEQ ID NO:8 beginning with the R or arginine at position 28. In one embodiment, the extracellular domain has the first 10 amino acids present at the amino terminal portion of the extracellular portion of the mature protein and accordingly comprises consecutive amino acids having the sequence set forth in SEQ ID NO:8 beginning with the C or cysteine at position 18.

In one embodiment, the thioredoxin comprises consecutive amino acids having the sequence set forth in SEQ ID NO:4 from the M or methionine at position 1 to the A or alanine at position 109. In one embodiment, the cDNA sequence which encodes the extracellular domain comprises consecutive nucleotides having the sequence set forth in SEQ ID NO:3 from the A or adenosine at position 502 to the G or guanosine at position 1503. In one embodiment, the cDNA sequence which encodes thioredoxin is set forth in SEQ ID NO:3 from the A or adenosine at position 1 to the C or cytosine at position 327.

The cDNA sequence for the full length human follicle stimulating hormone receptor is set forth in SEQ ID NO:7.

The amino acid sequence for the full length human follicle stimulating hormone receptor is set forth in SEQ ID NO:8.

As used herein “human follicle stimulating hormone” may be abbreviated as hFSH.

In one embodiment of the above nucleic acids, the peptide segment comprising consecutive histidine residues comprises at least 4 consecutive histidine residues. In one embodiment, the peptide segment comprising consecutive histidine residues comprises at least 6 consecutive histidine residues. In one embodiment, the peptide segment comprising consecutive histidine residues comprises at least 8 consecutive histidine residues.

In one embodiment, the nucleic acid is DNA. In one embodiment the DNA is cDNA. In one embodiment, the DNA is genomic DNA. In one embodiment, the nucleic acid is RNA. The nucleic acids of the subject invention may be isolated. The nucleic acids and polypeptides of the subject invention may be isolated. The nucleic acids and polypeptides of the subject invention may be purified.

As used herein, the following standard abbreviations are used throughout the specification to indicate specific amino acids: A=ala=alanine; R=arg=arginine; N=asn=asparagine; D=asp=aspartic acid; C=cys=cysteine; Q=gln=glutamine; E=glu=glutamic acid; G=gly=glycine; H=his=histidine; I=ile=isoleucine; L=leu=leucine; K=lys=lysine; M=met=methionine; F=phe=phenylalanine P=pro=proline; S=ser=serine; T=thr=threonine; W=trp=tryptophan; Y=tyr=tyrosine; and V=val=valine.

As used herein, the following standard abbreviations are used throughout the specification to indicate specific nucleotides: C=cytosine; A=adenosine; T=thymidine; G=guanosine; and U=uracil.

This invention provides a nucleic acid of at least 15 nucleotides in length capable of specifically hybridizing with the nucleic acids of the subject invention. The nucleic acid may be either DNA or RNA. The nucleic acid may be detectable. The nucleic acid may be labeled with a detectable marker. The detectable marker may be a radioactive, a colorimetric, a luminescent, or a fluorescent label.

High stringency hybridization conditions are selected at about 5° C. lower than the thermal melting point (Tₘ) for the specific sequence at a defined ionic strength and pH. The Tₘ is the temperature (under defined ionic strength and pH) at which 50% of the salt concentration is at least about 0.02 molar at pH 7 and the temperature is at least about 60° C. As other factors may significantly affect the stringency of hybridization, including, among others, base composition and size of the complementary strands, the presence of organic solvents, i.e. salt or formamide concentration, and the extent of base mismatching, the combination of parameters is more important than the absolute measure of any one. For example, high stringency may be attained by overnight hybridization at about 68° C. in a 6× SSC solution, washing at room temperature with 6× SSC solution, followed by washing at about 68° C. in a 0.6× SSC solution.

Hybridization with moderate stringency may be attained for example by: 1) filter pre-hybridizing and hybridizing with a solution of 3× SSC, 50% formamide, 0.1M Tris buffer at pH 7.5, 5× Denhardt's solution; 2.) pre-hybridization at 37° C. for 4 hours; 3) hybridization at 37° C. with amount of labeled probe equal to 3,000,000 cpm total for 16 hours; 4) wash in 2× SSC and 0.1% SDS solution; 5) wash 4× for 1 minute each at room temperature in 4× SSC at 60° C. for 30 minutes each; and 6) dry and expose to film.

The nucleic acids of the subject invention also include nucleic acids coding for polypeptide analogs, fragments or derivatives which differ from the naturally-occurring forms in terms of the identity of one or more amino acid residues (deletion analogs containing less than all of the specified residues; substitution analogs wherein one or more residues are replaced by one or more residues; and addition analogs, wherein one or more residues are added to a terminal or medial portion of the polypeptide) which share some or all of the properties of the naturally-occurring forms.

The nucleic acids, polypeptides and antibodies of the subject invention may be isolated and/or purified. One skilled in the art would know how to isolate or purify them.

This invention provides a replicable vector which comprises any one of the nucleic acids of the subject invention. In one embodiment, the vector is a plasmid, cosmid, λ phage or YAC. This invention provides a host cell which comprises the vector of the subject invention. In one embodiment, the cell is a bacterial cell. In one embodiment the bacterial cell is E. coli. In one embodiment, the cell comprises a thioredoxin reductase mutation. The cell may also or additionally comprise a glutathione reductase mutation. In one embodiment, the cell is a eukaryotic cell.

This invention provides a host-vector system for the production of a soluble polypeptide which comprises a vector of the subject invention and a suitable host cell.

This invention provides a method for producing a soluble polypeptide which comprises growing the host vector system of the subject invention under conditions permitting production of the soluble polypeptide and recovering the soluble polypeptide so produced.

This invention provides a soluble polypeptide encoded by a nucleic acid of the subject invention.

This invention provides a soluble polypeptide which comprises an extracellular domain of a gonadotropin receptor and thioredoxin, wherein the soluble polypeptide is capable of binding to the gonadotropin.

This invention provides a soluble polypeptide which comprises an extracellular domain of a gonadotropin receptor and a peptide segment comprising consecutive histidine residues, wherein the soluble polypeptide is capable of binding to the gonadotropin.

This invention provides a soluble polypeptide which comprises an extracellular domain of a gonadotropin receptor and thioredoxin and a peptide segment comprising consecutive histidine residues, wherein the soluble polypeptide is capable of binding to the gonadotropin.

In one embodiment of the soluble polypeptide, the gonadotropin receptor is a human luteinizing hormone/choriogonadotropin receptor and the soluble polypeptide is capable of binding to human luteinizing hormone or human chorionic gonadotropin.

In one embodiment of the soluble polypeptide, the gonadotropin receptor is a human follicle stimulating hormone receptor and the soluble polypeptide is capable of binding to human follicle stimulating hormone.

This invention provides a method of identifying an antibody capable of binding to an extracellular domain of a gonadotropin receptor which comprises:

(a) administering a polypeptide of the subject invention to a subject and obtaining antiserum from the subject;

(b) contacting a gonadotropin receptor with the antiserum;

(c) determining whether any antibody present in the antiserum binds to the a gonadotropin receptor and isolating such antibody, so as to thereby identify an antibody capable of binding to the extracellular domain of a gonadotropin receptor.

This invention provides a method of obtaining a composition which comprises:

(a) identifying an antibody capable of binding to an extracellular domain of a gonadotropin receptor by the above method; and

(b) admixing the antibody so identified with a carrier.

The assay may be carried out wherein one of the components is bound or affixed to a solid surface. In one embodiment the peptide is affixed to a solid surface. The solid surfaces useful in this embodiment would be known to one of skill in the art. For example, one embodiment of a solid surface is a bead, a column, a plastic dish, a plastic plate, a microscope slide, a nylon membrane, etc. The material of which the solid surface is comprised is synthetic in one example.

The assay may be carried out in vitro, wherein one or more of the components are attached or affixed to a solid surface, or wherein the components are admixed inside of a cell; or wherein the components are admixed inside of an organism (i.e. a transgenic mouse). For example, the peptide may be affixed to a solid surface. The antibody or the fragment thereof is affixed to a solid surface in another embodiment.

This invention provides a method of preventing a subject from becoming pregnant which comprises administering to the subject an amount of an antibody effective to bind to an extracellular domain of a gonadotropin receptor so as to thereby prevent the subject from becoming pregnant.

This invention provides a method of preventing a subject from becoming pregnant which comprises administering to the subject an amount of a polypeptide of the subject invention or an extracellular domain of a gonadotropin receptor so as to thereby prevent a subject from becoming pregnant.

This invention provides a method of terminating a pregnancy in a subject which comprises administering to the subject an amount of an antibody effective to bind to an extracellular domain of a gonadotropin receptor so as to thereby terminate the pregnancy in the subject.

This invention provides a method of terminating a pregnancy in a subject which comprises administering to the subject an amount of a polypeptide of the subject invention or an extracellular domain of a gonadotropin receptor so as to thereby terminate the pregnancy in the subject.

This invention provides a method of stimulating or enhancing production of an antibody capable of binding to an extracellular domain of a gonadotropin receptor in a subject which comprises administering to the subject an amount of a polypeptide of the subject invention or an extracellular domain of a gonadotropin receptor effective to stimulate or enhance antibody production in the subject.

This invention provides a method of treating a cancer in a subject which comprises administering to the subject an amount of a polypeptide of the subject invention or an extracellular domain of a gonadotropin receptor effective to stimulate or enhance production of an antibody capable of binding to an extracellular domain of a gonadotropin receptor so as to thereby treat the cancer in the subject.

This invention provides a method of treating a cancer in a subject which comprises administering to the subject an amount of an antibody effective to bind to an extracellular domain of a gonadotropin receptor so as to thereby treat a cancer in the subject.

This invention provides a method of preventing a cancer in a subject which comprises administering to the subject an amount of a polypeptide of the subject invention or an extracellular domain of a gonadotropin receptor effective to stimulate or enhance production of an antibody capable of binding to an extracellular domain of a gonadotropin receptor so as to thereby prevent the cancer in the subject.

This invention provides a method of preventing a cancer in a subject which comprises administering to the subject an amount of an antibody effective to bind to an extracellular domain of a gonadotropin receptor so as to thereby prevent a cancer in the subject. The cancers include but are not limited to lung cancer, bladder cancer, prostate cancer, colorectral cancer, ovarian cancer, cervical cancer, squamous cell cancer, or breast cancer.

This invention provides a method of decreasing a subject's production of androgen which comprises administering to the subject an amount of a polypeptide of the subject invention or an extracellular domain of a gonadotropin receptor effective to stimulate production of an antibody capable of binding to an extracellular domain of a gonadotropin receptor in the subject, so as to thereby decrease the subject's production of androgen.

This invention provides a method of decreasing a subject's production of androgen which comprises administering to the subject an amount of an antibody effective to bind to the extracellular domain of a gonadotropin receptor so as to thereby decrease the subject's production of androgen.

This invention provides a method of preventing a subject from becoming afflicted with ovarian hyperstimulatory syndrome which comprises administering to the subject an amount of an antibody effective to bind to an extracellular domain of a gonadotropin receptor so as to thereby prevent the subject from becoming afflicted with ovarian hyperstimulatory syndrome.

This invention provides a method of treating a subject afflicted with ovarian hyperstimulatory syndrome which comprises administering to the subject an amount of an antibody effective to bind to an extracellular domain of a gonadotropin receptor so as to thereby treat the subject afflicted with ovarian hyperstimulatory syndrome.

In one embodiment of the above methods, the gonadotropin receptor is human luteinizing hormone receptor, which may also referred to as human luteinizing hormone/choriogonadotropin receptor.

In one embodiment of the above methods, the gonadotropin receptor is follicle stimulating hormone receptor.

As used herein, “subject” includes any animal or artificially modified animal including but not limited to SCID mice with human immune systems. The animals include but are not limited to mice, rats, dogs, cats, guinea pigs, ferrets, rabbits, and primates. In the preferred embodiment, the subject is a human.

As used herein, “administering” may be effected or performed using any of the methods known to one skilled in the art, which includes intralesional, intraperitoneal, intramuscular, subcutaneous, intravenous, liposome mediated delivery, transmucosal, intestinal, topical, nasal, oral, anal, ocular or otic delivery.

The dose of the composition, antibody or polypeptide of the invention will vary depending on the subject and upon the particular route of administration used. Dosages can range from 0.1 to 100,000 μg/kg. Based upon the composition, the dose can be delivered continuously, such as by continuous pump, or at periodic intervals. For example, on one or more separate occasions. Desired time intervals of multiple doses of a particular composition can be determined without undue experimentation by one skilled in the art.

As used herein, “effective dose” means an amount in sufficient quantities to accomplish the specific task. A person of ordinary skill in the art can perform simple titration experiments to determine what amount is required to, for example, treat the subject.

In one embodiment, the effective amount of the composition comprises from about 0.000001 mg/kg body weight to about 100 mg/kg body weight of the subject.

This invention also provides for pharmaceutical compositions including therapeutically effective amounts of polypeptide compositions and compounds, together with suitable diluents, preservatives, solubilizers, emulsifiers, adjuvants and/or carriers. Such compositions may be liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the compound, complexation with metal ions, or incorporation of the compound into or onto particulate preparations of polymeric compounds such as polylactic acid, polglycolic acid, hydrogels, etc, or onto liposomes, micro emulsions, micelles, unilamellar or multi lamellar vesicles, erythrocyte ghosts, or spheroplasts. Such compositions will influence the physical state, solubility, stability, rate of in vivo release, and rate of in vivo clearance of the compound or composition. The choice of compositions will depend on the physical and chemical properties of the compound.

In the practice of any of the methods of the invention or preparation of any of the pharmaceutical compositions a “therapeutically effective amount” is an amount which is capable of preventing interaction of the receptor to the ligand in a subject. Accordingly, the effective amount will vary with the subject being treated, as well as the condition to be treated.

Controlled or sustained release compositions include formulation in lipophilic depots (e.g., fatty acids, waxes, oils). Also comprehended by the invention are particulate compositions coated with polymers (e.g., poloxamers or poloxamines) and the compound coupled to antibodies directed against tissue-specific receptors, ligands or antigens or coupled to ligands of tissue-specific receptors. Other embodiments of the compositions of the invention incorporate particulate forms protective coatings, protease inhibitors or permeation enhancers for various routes of administration, including parenteral, pulmonary, nasal and oral.

When administered, compounds are often cleared rapidly from the circulation and may therefore elicit relatively short-lived pharmacological activity. Consequently, frequent injections of relatively large doses of bioactive compounds may by required to sustain therapeutic efficacy. Compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound adducts less frequently or in lower doses than with the unmodified compound.

Attachment of polyethylene glycol (PEG) to compounds is particularly useful because PEG has very low toxicity in mammals (Carpenter et al., 1971). For example, a PEG adduct of adenosine deaminase was approved in the United States for use in humans for the treatment of severe combined immunodeficiency syndrome. A second advantage afforded by the conjugation of PEG is that of effectively reducing the immunogenicity and antigenicity of heterologous compounds. For example, a PEG adduct of a human protein might be useful for the treatment of disease in other mammalian species without the risk of triggering a severe immune response. The polypeptide or composition of the present invention may be delivered in a microencapsulation device so as to reduce or prevent an host immune response against the polypeptide or against cells which may produce the polypeptide. The polypeptide or composition of the present invention may also be delivered microencapsulated in a membrane, such as a liposome.

Polymers such as PEG may be conveniently attached to one or more reactive amino acid residues in a protein such as the alpha-amino group of the amino terminal amino acid, the epsilon amino groups of lysine side chains, the sulfhydryl groups of cysteine side chains, the carboxyl groups of aspartyl and glutamyl side chains, the alpha-carboxyl group of the carboxy-terminal amino acid, tyrosine side chains, or to activated derivatives of glycosyl chains attached to certain asparagine, serine or threonine residues.

Numerous activated forms of PEG suitable for direct reaction with proteins have been described. Useful PEG reagents for reaction with protein amino groups include active esters of carboxylic acid or carbonate derivatives, particularly those in which the leaving groups are N-hydroxysuccinimide, p-nitrophenol, imidazole or 1-hydroxy-2-nitrobenzene-4-sulfonate. PEG derivatives containing maleimido or haloacetyl groups are useful reagents for the modification of protein free sulfhydryl groups. Likewise, PEG reagents containing amino hydrazine or hydrazide groups are useful for reaction with aldehydes generated by periodate oxidation of carbohydrate groups in proteins.

In one embodiment, the pharmaceutical carrier may be a liquid and the pharmaceutical composition would be in the form of a solution. In another equally preferred embodiment, the pharmaceutically acceptable carrier is a solid and the composition is in the form of a powder or tablet. In a further embodiment, the pharmaceutical carrier is a gel and the composition is in the form of a suppository or cream. In a further embodiment the active ingredient may be formulated as a part of a pharmaceutically acceptable transdermal patch.

A solid carrier can include one or more substances which may also act as flavoring agents, lubricants, solubilizers, suspending agents, fillers, glidants, compression aids, binders or tablet-disintegrating agents; it can also be an encapsulating material. In powders, the carrier is a finely divided solid which is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, polyvinylpyrrolidine, low melting waxes and ion exchange resins.

Liquid carriers are used in preparing solutions, suspensions, emulsions, syrups, elixirs and pressurized compositions. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also be an oily ester such as ethyl oleate and isopropyl myristate. Sterile liquid carriers are useful in sterile liquid form compositions for parenteral administration. The liquid carrier for pressurized compositions can be halogenated hydrocarbon or other pharmaceutically acceptable propellent.

Liquid pharmaceutical compositions which are sterile solutions or suspensions can be utilized by for example, intramuscular, intrathecal, epidural, intraperitoneal or subcutaneous injection. Sterile solutions can also be administered intravenously. The active ingredient may be prepared as a sterile solid composition which may be dissolved or suspended at the time of administration using sterile water, saline, or other appropriate sterile injectable medium. Carriers are intended to include necessary and inert binders, suspending agents, lubricants, flavorants, sweeteners, preservatives, dyes, and coatings.

The active ingredient of the present invention (i.e., the compound identified by the screening method or composition thereof) can be administered orally in the form of a sterile solution or suspension containing other solutes or suspending agents, for example, enough saline or glucose to make the solution isotonic, bile salts, acacia, gelatin, sorbitan monoleate, polysorbate 80 (oleate esters of sorbitol and its anhydrides copolymerized with ethylene oxide) and the like.

The active ingredient can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

When administered orally or topically, such agents and pharmaceutical compositions would be delivered using different carriers. Typically such carriers contain excipients such as starch, milk, sugar, certain types of clay, gelatin, stearic acid, talc, vegetable fats or oils, gums, glycols, or other known excipients. Such carriers may also include flavor and color additives or other ingredients. The specific carrier would need to be selected based upon the desired method of deliver, e.g., PBS could be used for intravenous or systemic delivery and vegetable fats, creams, salves, ointments or gels may be used for topical delivery.

This invention also provides for pharmaceutical compositions including therapeutically effective amounts of protein compositions and/or agents capable of inhibiting the binding of a ligand with a receptor in the subject of the invention together with suitable diluents, preservatives, solubilizers, emulsifiers, adjuvants and/or carriers. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the agent, complexation with metal ions, or incorporation of the agent into or onto particulate preparations of polymeric agents such as polylactic acid, polglycolic acid, hydrogels, etc, or onto liposomes, micro emulsions, micelles, unilamellar or multi lamellar vesicles, erythrocyte ghosts, or spheroplasts. Such compositions will influence the physical state, solubility, stability, rate of in vivo release, and rate of in vivo clearance of the agent or composition. The choice of compositions will depend on the physical and chemical properties of the agent capable of alleviating the symptoms in the subject.

The agent of the present invention may be delivered locally via a capsule which allows sustained release of the agent or the peptide over a period of time. Controlled or sustained release compositions include formulation in lipophilic depots (e.g., fatty acids, waxes, oils). Also comprehended by the invention are particulate compositions coated with polymers (e.g., poloxamers or poloxamines) and the agent coupled to antibodies directed against tissue-specific receptors, ligands or antigens or coupled to ligands of tissue-specific receptors. Other embodiments of the compositions of the invention incorporate particulate forms protective coatings, protease inhibitors or permeation enhancers for various routes of administration, including parenteral, pulmonary, nasal and oral.

In one embodiment, the carrier comprises a diluent. In another embodiment, the carrier comprises, a virus, a liposome, a microencapsule, a polymer encapsulated cell or a retroviral vector. In another embodiment, the carrier is an aerosol, intravenous, oral or topical carrier, or aqueous or nonaqueous solution. For example, the compound is administered from a time release implant.

As used herein, the term “suitable pharmaceutically acceptable carrier” encompasses any of the standard pharmaceutically accepted carriers, such as phosphate buffered saline solution, water, emulsions such as an oil/water emulsion or a triglyceride emulsion, various types of wetting agents, tablets, coated tablets and capsules. An example of an acceptable triglyceride emulsion useful in intravenous and intraperitoneal administration of the compounds is the triglyceride emulsion commercially known as Intralipid®.

Typically such carriers contain excipients such as starch, milk, sugar, certain types of clay, gelatin, stearic acid, talc, vegetable fats or oils, gums, glycols, or other known excipients. Such carriers may also include flavor and color additives or other ingredients.

The compounds, agents, peptides, antibodies, and fragments thereof of the present invention may be detectably labeled. The detectable label may be a fluorescent label, a biotin, a digoxigenin, a radioactive atom, a paramagnetic ion, and a chemiluminescent label. It may also be labeled by covalent means such as chemical, enzymatic or other appropriate means with a moiety such as an enzyme or radioisotope. Portions of the above mentioned compounds of the invention may be labeled by association with a detectable marker substance (e.g., radiolabeled with ²²⁵I or biotinylated) to provide reagents useful in detection and quantification of compound or its receptor bearing cells or its derivatives in solid tissue and fluid samples such as blood, cerebral spinal fluid or urine.

The present invention also provides for a transgenic nonhuman mammal whose germ or somatic cells contain a nucleic acid molecule which encodes the receptor or fragment thereof or a biologically active variant thereof, introduced into the mammal, or an ancestor thereof, at an embryonic stage. In one embodiment, the nucleic acid molecule which encodes the receptor or fragment thereof is overexpressed in the cells of the mammal.

The present invention also provides for a transgenic nonhuman mammal whose germ or somatic cells have been transfected with a suitable vector with an appropriate sequence designed to reduce expression levels of the receptor below the expression levels of that of a native mammal. In one embodiment, the suitable vector contains an appropriate piece of cloned genomic nucleic acid sequence to allow for homologous recombination. In another embodiment, the suitable vector encodes a ribozyme capable of cleaving a receptor mRNA molecule or an antisense molecule which comprises a sequence antisense to naturally occurring mRNA sequence.

The present invention provides for a transgenic non-human animal whose cells contain a DNA sequence comprising: (a) a nerve tissue specific promoter; and (b) a DNA sequence which encodes a polypeptide, wherein the promoter and the DNA sequence which encodes the polypeptide are operatively linked to each other and integrated in the genome of the non-human animal, and wherein said nonhuman animal exhibits a phenotype which differs from that of an identical non-human animal lacking said DNA sequence.

The “transgenic non-human animals” of the invention may be produced by introducing “transgenes” into the germline of the non-human animal.

Transgenic Technology and Methods: the following U.S. patents are hereby incorporated by reference: U.S. Pat. No. 6,025,539, IL-5 transgenic mouse; U.S. Pat. No. 6,023,010, Transgenic non-human animals depleted in a mature lymphocytic cell-type; U.S. Pat. No. 6,018,098, In vivo and in vitro model of cutaneous photoaging; U.S. Pat. No. 6,018,097, Transgenic mice expressing human insulin; U.S. Pat. No. 6,008,434, Growth differentiation factor-11 transgenic mice; U.S. Pat. No. 6,002,066; H2-M modified transgenic mice; U.S. Pat. No. 5,994,618, Growth differentiation factor-8 transgenic mice; U.S. Pat. No. 5,986,171, Method for examining neurovirulence of polio virus, U.S. Pat. No. 5,981,830, Knockout mice and their progeny with a disrupted hepsin gene; U.S. Pat. No. 5,981,829, .DELTA.Nur77 transgenic mouse; U.S. Pat. No. 5,936,138; Gene encoding mutant L3T4 protein which facilitates HIV infection and transgenic mouse expressing such protein; U.S. Pat. No. 5,912,411, Mice transgenic for a tetracycline-inducible transcriptional activator; U.S. Pat. No. 5,894,078, Transgenic mouse expressing C-100 app.

The methods used for generating transgenic mice are well known to one of skill in the art. For example, one may use the manual entitled “Manipulating the Mouse Embryo” by Brigid Hogan et al. (Ed. Cold Spring Harbor Laboratory) 1986.

See for example, Leder and Stewart, U.S. Pat. No. 4,736,866 for methods for the production of a transgenic mouse.

For sometime it has been known that it is possible to carry out the genetic transformation of a zygote (and the embryo and mature organism which result therefrom) by the placing or insertion of exogenous genetic material into the nucleus of the zygote or to any nucleic genetic material which ultimately forms a part of the nucleus of the zygote. The genotype of the zygote and the organism which results from a zygote will include the genotype of the exogenous genetic material. Additionally, the inclusion of exogenous genetic material in the zygote will result in a phenotype expression of the exogenous genetic material.

The genotype of the exogenous genetic material is expressed upon the cellular division of the zygote. However, the phenotype expression, e.g., the production of a protein product or products of the exogenous genetic material, or alterations of the zygote's or organism's natural phenotype, will occur at that point of the zygote's or organism's development during which the particular exogenous genetic material is active. Alterations of the expression of the phenotype include an enhancement or diminution in the expression of a phenotype or an alteration in the promotion and/or control of a phenotype, including the addition of a new promoter and/or controller or supplementation of an existing promoter and/or controller of the phenotype.

The genetic transformation of various types of organisms is disclosed and described in detail in U.S. Pat. No. 4,873,191, issued Oct. 10, 1989, which is incorporated herein by reference to disclose methods of producing transgenic organisms. The genetic transformation of organisms can be used as an in vivo analysis of gene expression during differentiation and in the elimination or diminution of genetic diseases by either gene therapy or by using a transgenic non-human mammal as a model system of a human disease. This model system can be used to test putative drugs for their potential therapeutic value in humans.

The exogenous genetic material can be placed in the nucleus of a mature egg. It is preferred that the egg be in a fertilized or activated (by parthenogenesis) state. After the addition of the exogenous genetic material, a complementary haploid set of chromosomes (e.g., a sperm cell or polar body) is added to enable the formation of a zygote. The zygote is allowed to develop into an organism such as by implanting it in a pseudopregnant female. The resulting organism is analyzed for the integration of the exogenous genetic material. If positive integration is determined, the organism can be used for the in vivo analysis of the gene expression, which expression is believed to be related to a particular genetic disease.

Attempts have been made to study a number of different types of genetic diseases utilizing such transgenic animals. Attempts related to studying Alzheimer's disease are disclosed within published PCT application WO89/06689 and PCT application WO89/06693, both published on Jul. 27, 1989, which published applications are incorporated herein by reference to disclose genetic sequences coding for Alzheimer's .beta.-amyloid protein and the incorporation of such sequences into the genome of transgenic animals.

Embryonal target cells at various developmental stages can be used to introduce transgenes. Different methods are used depending on the stage of development of the embryonal target cell. The zygote is the best target for micro-injection. In the mouse, the male pronucleus reaches the size of approximately 20 micrometers in diameter which allows reproducible injection of 1-2 pl of DNA solution. The use of zygotes as a target for gene transfer has a major advantage in that in most cases the injected DNA will be incorporated into the host gene before the first cleavage (Brinster, et al. (1985) Proc. Natl. Acad. Sci. U.S.A. 82, 4438-4442). As a consequence, all cells of the transgenic non-human animal will carry the incorporated transgene. This will in general also be reflected in the efficient transmission of the transgene to offspring of the founder since 50% of the germ cells will harbor the transgene. Microinjection of zygotes is the preferred method for incorporating transgenes in practicing the invention.

Retroviral infection can also be used to introduce transgene into a non-human animal. The developing nonhuman embryo can be cultured in vitro to the blastocyst stage. During this time, the blastomeres can be targets for retroviral infection (Jaenich, R. (1976) Proc. Natl. Acad. Sci U.S.A. 73, 1260-1264). Efficient infection of the blastomeres is obtained by enzymatic treatment to remove the zona pellucida (Hogan, et al. (1986) in Manipulating the Mouse Embryo, Cold Spring Harbor Laboratory Press, Cold Spring Harbor, N.Y.). The viral vector system used to introduce the transgene is typically a replication-defective retrovirus carrying the transgene (Jahner, et al. (1985) Proc. Natl. Acad. Sci. U.S.A. 82, 6927-6931; Van der Putten, et al. (1985) Proc. Natl. Acad. Sci U.S.A. 82, 6148-6152). Transfection is easily and efficiently obtained by culturing the blastomeres on a monolayer of virus-producing cells (Van der Putten, supra; Stewart, et al. (1987) EMBO J. 6, 383-388). Alternatively, infection can be performed at a later stage. Virus or virus-producing cells can be injected into the blastocoele (Jahner, D., et al. (1982) Nature 298, 623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line, albeit with low efficiency, by intrauterine retroviral infection of the midgestation embryo (Jahner, D. et al. (1982) supra).

A third type of target cell for transgene introduction is the embryonal stem cell (ES). ES cells are obtained from pre-implantation embryos cultured in vitro and fused with embryos (Evans, M. J., et al. (1981) Nature 292, 154-156; Bradley, M. 0., et al. (1984) Nature 309, 255-258; Gossler, et al. (1986) Proc. Natl. Acad. Sci U.S.A. 83, 9065-9069; and Robertson, et al. (1986) Nature 322, 445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240, 1468-1474.

As used herein, a “transgene” is a DNA sequence introduced into the germline of a non-human animal by way of human intervention such as by way of the above described methods.

A number of cancers are known to produce hCG, inlcuding testicular, prostatic, colon, pancreatic, lung, trophoblastic, ovarian and cervical cancers (41). In vitro, hCG stimulates proliferation of neoplastic cells (42) and confers resistence to chemotherapy-induced cell death. Expression of hCG in vivo has been associated with more aggressive histology (43), more advanced stage of disease (44), resistence to chemoterapy (44), and a worse prognosis (45). Antibodies to hCG have been effective in inhibiting growth of cancer cells, and have demonstrated cell cytotoxicity (46,47). Clinical trials using vaccines directed against hCG are currently ongoing for a number of malihnancies (AVI therapeutics).

In women, a primary stimulus for ovarian androgen output from theca cells of the ovary is luteinizing hormone (hLH). In men, the primary stimulus for testicular androgen from the leydig cells is hLH. In fetal life, a primary stimulus for testicular androgen productions is hCG.

FSH induces ovarian follicular development, culminating in the selection of dominant follicle and subsequent ovulation. As follicles grow, production of estradiol by granulosa cells increases. Serum levels of estradiol thus increase from a baseline (early follicular) level of 30-50 pg/mL to 250-300 pg/mL in the late follicular phase just prior to ovulation. Pharmacologic doses of FSH are used to induce multifollicular maturation in women undergoing assisted reproduction with serum estradiol levels reaching a peak of 1000-4000 pg/mL or higher (200-250 pg/mL/mature follicle).

Another potential application for the hCG and hFSH receptor extracellular domains is the following. Ovarian hyperstimulation syndrome may be prevented by withholding the HCG injection during controlled ovarian hyperstimulation cycles. Some clinicians will reduce the dose of hCG (from 10,000 units to 5,000 units) when concerned about the risk for hyperstimulation in a particular patient. An inhibitor of the hCG receptor can prevent or reverse this untoward effect (in egg donors, any pregnancy-related concerns are irrelevant). An hFSH receptor antagonist can also prevent this complication (it is clearly initiated by overzealous hFSH stimulation in the first place).

In one embodiment, the antireceptor antibody is a monoclonal antibody. In one embodiment, the monoclonal antibody is a human, humanized or chimeric antibody. In one embodiment, the portion of the antibody is a Fab fragment of the antibody. In one embodiment, the portion of the antibody comprises the variable domain of the antibody. In one embodiment, the portion of the antibody comprises a CDR portion of the antibody. In one embodiment, the monoclonal antibody is an IgG, IgM, IgD, IgA, or IgE monoclonal antibody.

As used herein, “antibody” means an immunoglobulin molecule comprising two heavy chains and two light chains and which recognizes an antigen. The immunoglobulin molecule may derive from any of the commonly known classes, including but not limited to IgA, secretory IgA, IgG and IgM. IgG subclasses are also well known to those in the art and include but are not limited to human IgG1, IgG2, IgG3 and IgG4. It includes, by way of example, both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man. Methods for humanizing antibodies are known to those skilled in the art.

This invention provides humanized forms of the above antibodies. As used herein, “humanized” describes antibodies wherein some, most or all of the amino acids outside the complementarity determining region (“CDR”) regions are replaced with corresponding amino acids derived from human immunoglobulin molecules. In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody would retain a similar antigenic specificity as the original antibody.

One skilled in the art would know how to make the humanized antibodies of the subject invention. Various publications, several of which are hereby incorporated by reference into this application, also describe how to make humanized antibodies. For example, the methods described in U.S. Pat. No. 4,816,567 (57) comprise the production of chimeric antibodies having a variable region of one antibody and a constant region of another antibody.

U.S. Pat. No. 5,225,539 (58) describes another approach for the production of a humanized antibody. This patent describes the use of recombinant DNA technology to produce a humanized antibody wherein the CDRs of a variable region of one immunoglobulin are replaced with the CDRs from an immunoglobulin with a different specificity such that the humanized antibody would recognize the desired target but would not be recognized in a significant way by the human subject's immune system. Specifically, site directed mutagenesis is used to graft the CDRs onto the framework.

Other approaches for humanizing an antibody are described in U.S. Pat. Nos. 5,585,089 (59) and 5,693,761 (60) and WO 90/07861 which describe methods for producing humanized immunoglobulins. These have one or more CDRs and possible additional amino acids from a donor immunoglobulin and a framework region from an accepting human immunoglobulin. These patents describe a method to increase the affinity of an antibody for the desired antigen. Some amino acids in the framework are chosen to be the same as the amino acids at those positions in the donor rather than in the acceptor. Specifically, these patents describe the preparation of a humanized antibody that binds to a receptor by combining the CDRs of a mouse monoclonal antibody with human immunoglobulin framework and constant regions. Human framework regions can be chosen to maximize homology with the mouse sequence. A computer model can be used to identify amino acids in the framework region which are likely to interact with the CDRs or the specific antigen and then mouse amino acids can be used at these positions to create the humanized antibody.

The above U.S. Pat. Nos. 5,585,089 and 5,693,761, and WO 90/07861 (61) also propose four possible criteria which may be used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies.

This invention also provides for the construction and expression of gonadotropin receptor chimeras fused to thioredoxin. These include hLH/CG and hFSH receptors. Expression of a hLH/CG:hFSH receptor chimera fusion is useful for the production of a protein that generates an immune response targeted against both the hFSH and hLH/CG receptors. In addition, chimeras are useful for structural studies to pinpoint the structural nuances that are involved in the specificity of these receptors for their respective ligands. To produce these chimeras, a part of one receptor is swapped for the homologous region of the other receptor. To this end, the extracellular domain of each receptor is divided into four contiguous segments consisting on average of about 25% of the extracellular domain of the receptor. Accordingly, each segment of a receptor would be about 84 amino acid residues in length (i.e. about 25% of the extracellular domain). Alternatively, a segment may be greater than 25% of the extracellular domain, such as about 20%, about 21%, about 22%, about 23% or about 24%. Alternatively, a segment may be less than 25% of the extracellular domain, such as about 26%, about 27%, about 28%, about 29% or about 30% of the extracellular domain.

Accordingly, in the chimera, a segment may alternatively be one of the following lengths: 83 amino acids; 82 amino acids; 81 amino acids; 80 amino acids; 79 amino acids; 78 amino acids; 77 amino acids; 76 amino acids; 75 amino acids; 74 amino acids; 73 amino acids; 72 amino acids; 71 amino acids; or 70 amino acids. A segment may alternatively be shorter than 70 amino acids. A segment may alternatively be one of the following lengths: 85 amino acids; 86 amino acids; 87 amino acids; 88 amino acids; 89 amino acids; 90 amino acids; 91 amino acids; 92 amino acids; 93 amino acids; 94 amino acids; 95 amino acids; 96 amino acids; 97 amino acids; or 98 amino acids. A segment may alternatively be longer than 98 amino acids.

In the chimera, a segment of one receptor is then replaced with the homologous region of the other receptor. Accordingly, there are eight types of chimeras. Four types of chimeras comprise of 3 segments from the hLH/CG receptor and one segment from the hFSH receptor. The other four types of chimeras comprise 3 segments from the hFSH receptor and one segment of the hLH/CG receptor.

This invention is illustrated in the Experimental Details section which follows. These sections are set forth to aid in an understanding of the invention but are not intended to, and should not be construed to, limit in any way the invention as set forth in the claims which follow thereafter. One skilled in the art will readily appreciate that the specific methods and results discussed are merely illustrative of the invention as described more fully in the claims which follow thereafter.

### Experimental Details

A) Materials and Methods:

Bacterial Strains:

All molecular biology techniques and large scale preparation of plasmid DNA was performed with E. coli strain DH5α. Expression constructs were transformed into either strain AD494 trxB (DE3)pLys5 (kan^{R}, Cm^{R}) or Origami trxB gor (DE3)pLysS (kan^{R}, tet^{R}, Cm^{R}). Both strains were acquired from Novagen (Madison, Wis.) and are mutant at the thioredoxin reductase locus (trxB). The Origami strain is also mutant at the glutathione reductase locus and is designated gor. For expression of fusion constructs the strains were freshly transformed and colonies were picked directly from the transformation plates into growth media for expression. Passing or freezing transformants leads to diminished expression from the bacterial population. Strains were grown in standard Luria-Bertani medium (LB) for expression. Richer media formulations such as SOC media lead to poorer expression yields of soluble protein.

Gonadotropin Preparations:

The preparations of hCG (urinary hCG CR127) used in this study have previously been described (34,35). The CR127 preparation of hCG is the widely distributed reference preparation (35). Human FSH was acquired from the National Pituitary Agency (NIH).

Molecular Biology:

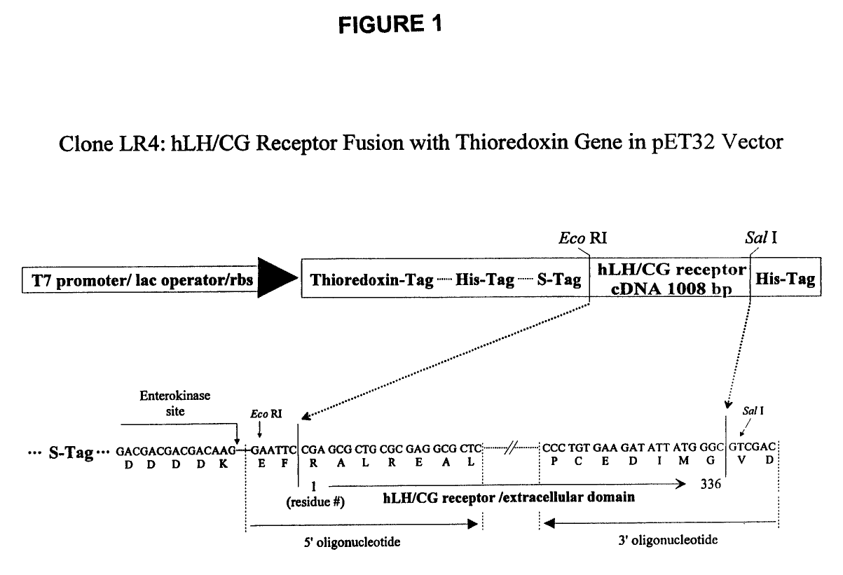

All enzymes for recombinant DNA were purchased from New England Biolabs. DNA primers for PCR were synthesized by the Columbia University Core Laboratory. The 5′ primer introduced an Eco RI site in the same frame and adjacent to the receptor sequence whereas the 3′ primer introduced an in frame Sal I site (see FIG. 1). PCR reactions were performed with Vent DNA polymerase (New England Biolabs) and all products of the reactions were sequenced to ensure that no mutations were introduced during the amplification process. Ligation reactions were transformed into DH5α and DNA clones were grown in DH5α for large scale plasmid preparation. Transformation of DH5α and the expression strains AD494 and Origami were performed according to standard techniques with calcium chloride.

Construction of Thioredoxin Fusion Clone:

The expression vector for these experiments was pET32a(+) (Novagen, Madison Wis.). DNA encoding the extracellular domain of the hLH/CG receptor was isolated by PCR of a full length clone of the hLH/CG receptor (kindly provided by Dr. Aaron Hsueh). In frame 5′ and 3′ Eco RI and Sal I sites respectively, were engineered into the amplified sequence through the 5′ and 3′ primers (see FIG. 1). Digestion with Eco RI and Sal I followed by ligation of this Eco RI-Sal I PCR product into the polylinker cloning site of the pET32a(+) vector generated a frame shift 3′ to the receptor sequence such that the carboxy terminal residues of this clone are equivalent to those encoded in the pET32b(+) clone. As a result a second His tag that is normally present in the pET32b(+) vector is also encoded at the 3′ end or carboxy terminal region of this clone (see FIG. 1). The clone utilized for all expression studies was designated LR4.

Expression of Fusion Protein:

For expression in AD494, colonies were inoculated into 10 mL of LB media with kanamycin (50 μg/mL) and ampicillin (75 μg/mL) and grown overnight. The fresh overnight culture was then diluted 1:100 into LB with 50 μg/mL ampicillin and grown to an OD of 0.5 at 600 nm. The culture was then induced with 1 mM isopropyl-thio-β-D-thiogalactoside (IPTG) for 3 hours and the bacteria were harvested by centrifugation. Bacterial pellets were then frozen and stored at 20° C. Expression in the Origami strain was essentially the same except that the overnight culture was grown in the presence of tetracycline (25 μg/mL) in addition to ampicillin and kanamycin.

Purification of Expressed Fusion Protein:

Frozen bacterial pellets were resuspended in 1 mL of Bugbuster reagent (Novagen, Madison Wis.) per 50 mL of bacterial culture with the addition of 20 μg/mL of lysozyme. The suspended cells were allowed to sit on ice until lysis was complete and the suspension became viscous. The lysed cells were then incubated with 25 units of Benzonase nuclease (Novagen, Madison Wis.) per mL. The mixture was incubated on ice until the viscosity disappeared. Aggregated protein was collected by further incubation of the material on ice for either 2-3 hours or overnight at 4° C. followed by centrifugation. The protein pellet was resuspended in 10 mM NaHPO₄/60 mM NaCl pH 8.0 at 1 mL/mL of suspended cells. N-Lauroylsarcosine was then added at a final concentration of 0.25% and the material was mixed well by pipetting up and down through the tip of an eppendorf pipette to help disperse the aggregated protein. The resuspended protein was then allowed to sit on ice until the solution clarified and solubilization was complete. If solubilization was not complete the solution was diluted twofold and additional N-Lauroylsarcosine was added to obtain a final concentration of 0.25%. Incomplete initial solubilization at 0.25% N-Lauroylsarcosine generally indicated that bacterial expression exceeded 20 mg/L and twofold dilution of the solution led to complete solubilization at 0.25% N-Lauroylsarcosine in all cases.

Solubilized fusion protein in 0.25% N-Lauroylsarcosine was diluted 10 fold in 50 mM NaHPO₄/300 mM NaCl pH 8.0 for a final detergent concentration of 0.025%. This material was passed through a Ni-NTA resin column (Qiagen) and the flow through was recirculated through the resin 5 times. The resin was then washed with five volumes of 50 mM NaHPO₄/300 mM NaCl pH 8.0 followed by the same buffer with the addition of 10 or 20 mM Imidiazole pH 8.0 for the second and third washes respectively. The bound fusion protein was then eluted with three column volumes of 50 mM NaHPO₄/300 mM NaCl/250 mM Imidazole pH 8.0. The eluted material was then exhaustively dialyzed in lOmM NaHPO₄pH 8.0.

Electrophoresis:

SDS-polyacrylamide gel electrophoresis was performed according to the procedure described by Laemmli (36,37). The sample buffer contained 125 mM Tris-HCl, pH 6.8, 10% glycerol, 2% SDS, 0.01% bromophenol blue. Gels were 10% polyacrylamide and either coomassie blue or silver stained according to established techniques (38).

Western Blot Analysis:

Following electrophoresis, the proteins in the gel were transferred to nitrocellulose paper using a variation of the methods of Towbin (39) and Burnette (40). After blocking in 5% BSA, 0.01M Tris-HCl, 0.15M NaCl pH 7.6 (BSA-TBS), the paper was incubated overnight with antibody diluted in BSA-TBS (at concentrations of approximately 3-4 ug antibody/ml for monoclonal antibodies and using a dilution of 1:500 of anti-hLH/CG receptor antiserum for polyclonal antibodies). The binding of the primary antibodies was visualized as previously described (17).

Monoclonal and Polyclonal Antibodies:

All monoclonal antibodies used in these studies have been previously described (13,17,18). B105 binds both intact hCG and free β-subunit. B105 does not block hCG from binding to the receptor and can bind hCG simultaneously with hCG binding to the receptor (5,17,18). Alternatively, B107 only binds intact hCG, cannot bind hCG while bound to receptor and blocks binding of free hCG to the receptor (5,17,18). Monoclonal LHR29 made against the hLH/CG receptor was obtained from E. Milgrom (13). Polyclonal antisera against the extracellular domain of the hLH/CG receptor was produced in the laboratory and previously described (5).

Determination of the Affinity of the Fusion Protein for hCG:

To determine the affinity of the fusion protein for hCG a reliable and highly reproducible technique for assaying binding using the His tag for capture of receptor bound material was developed. This was accomplished by using the Ni-NTA resin to capture receptor fusion protein following incubation with radiolabeled ¹²⁵I-hCG. Briefly, purified receptor material was incubated in 50 mM NaHPO₄/300 mM NaCl pH 8.0 with ¹²⁵I-hCG for 2-4 hours at ambient temperature or at 4° C. overnight. Incubation volume was standardized at 1 mL and approximately 200 ng of receptor fusion protein with 2×10⁵ cpm of labeled hCG (approximately 75 fm) was added to each incubation reaction. Ni-NTA resin was prepared by extensive washing with 50 mM NaHPO₄/300 mM NaCl pH 8.0 followed by resuspension in the same buffer with a volume equivalent to packed resin volume. Typically 20 μL of the resuspended resin was added to each incubation reaction and the suspension was incubated an additional 2 hours at ambient temperature with mixing every 10 minutes to resuspend the settled resin. The resin was then spun down and washed twice with 50 mM NaHPO₄/300 mM NaCl pH 8.0 followed by the same buffer including 20 mM Imidazole at pH 8.0. Bound hCG was then identified by counting in a gamma counter. Background generated by binding of the labeled hCG alone to the resin was generally less than 5% of that obtained when receptor material was added to the incubation.

To facilitate an accurate determination of affinity, 2×10⁵ cpm of ¹²⁵I-hCG was titrated with decreasing amounts of receptor material until the number of bound counts began to decrease. At that point the amount of receptor material was not in excess and was just sufficient to bind the maximum number of counts (i.e., as many counts as a saturating amount of receptor material). Using this same amount of receptor material (approximately 200 ng) with 2×10⁵ cpm of ¹²⁵I-hCG (approximately 75 fm) in each incubation reaction the affinity of the expressed receptor fusion for hCG was determined by adding decreasing amounts of cold hCG to each incubation reaction such that the concentration of cold hCG varied between 10⁻⁷ and 10⁻¹²M. The data was plotted as molarity of cold hCG versus % maximally bound ¹²⁵I-hCCG (see FIG. 3). Maximally bound ¹²⁵I-hCG is equal to the number of counts bound in the absence of cold hCG. The % maximally bound ¹²⁵I-hCG is then determined from the equation [cpm bound hCG in presence of test amount of cold hCG]/[cpm maximally bound hCG]×100. The % maximally bound ¹²⁵I-hCG is presented instead of absolute counts so that data could be readily compared from a series of binding experiments that may have employed different preparations of labeled hCG and receptor protein. Graphs were plotted with the program Axum 5.0 (Mathsoft, Cambridge, Mass.) and a best fit curve was drawn through the data points using the Lowess algorithm.

Effect of Monoclonal Antibodies B105 and B107 on hCG Binding to Receptor:

To test the effect of B105 or B107 on binding of labeled hCG to the receptor we titrated various dilutions of these antibodies with labeled hCG, in the binding assay described above, to determine an appropriate antibody dilution for these studies. High concentrations of a given monoclonal antibody will simulate binding since they bind the Ni-NTA resin nonspecifically and therefore bring down labeled hCG in the resin pellet. Therefore we chose as a working dilution of antibody a concentration that caused an increase of 10% or less in the background binding of hCG to the Ni-NTA resin (i.e., labeled hCG and resin). Addition of these dilutions of antibodies in the Ni-NTA binding assay had the desired effect of blocking binding to recombinant receptor in the case of B107 and having little or no effect in the case of B105 (see FIG. 4).

The extracellular domain of the hLH/CG receptor as a fusion protein with thioredoxin in the cytoplasm of an E. coli strain that contains mutations in both the thioredoxin reductase and glutathione reductase genes has been expressed. The chimeric protein isolated following induction of expression is purified in a soluble form and binds hCG with an affinity approximating that of native receptor. This truncated form of the receptor displays the same specificity as intact hLH/CG receptor and does not bind HFSH. This cytoplasmically produced protein is expressed at levels that exceed 10mg/L. Expression of properly folded extracellular domain of the hLH/CG receptor in the cytoplasm of E. coli allows the facile and economical purification of large quantities of material. This will facilitate the determination of the structure of the hormone binding domain of the glycoprotein receptor as well as the production of epitope specific antibodies.

B) Results

Construction of an Expression Clone Containing the Extracellular Domain of the hLH/CG Receptor:

To express the extracellular domain of the hLH/CG receptor in E. coli a number of different expression clones were constructed. Few of these showed significant expression upon induction. In particular, attempts to express the extracellular domain of the receptor in the cytoplasm of E. coli failed in a number of different strains tested. As a result it was concluded that those sequences did not fold properly or were perhaps unstable by themselves in the cytoplasm of E. coli. To address this problem cDNA encoding the extracellular domain of the hLH/CG receptor was fused with a number of different proteins that were known to enhance stability and promote folding of the tethered sequences. One clone, designated LR4, expressed significant amounts of material upon induction. It contained a fusion of the receptor sequences with the thioredoxin gene in a pET32a(+) vector and is the source of recombinant material described herein.

Clone LR4 contains residues 1-336 from the mature receptor protein. These residues encompass nearly the entire extracellular domain of the receptor. Using primers with engineered 5′ Eco RI and 3′ Sal I sites, a cDNA encoding residues 1-336 of the receptor was amplified by PCR. The amplified product contained in frame 5′ Eco RI and 3′ Sal I sites (see Material and Methods section and FIG. 1). This was inserted into the polylinker cloning site of the pET32a(+) vector from Novagen. The resulting construct has the receptor sequences fused downstream of the thioredoxin gene. In addition, the insertion generates a frameshift such that the 3′ residues at the carboxy terminus encoded in this clone are equivalent to those encoded by the pET32b(+) clone. These terminal residues in pET32b(+) comprise a second His tag. Therefore the LR4 fusion clone encodes a second His tag at the 3′ end with the other tag sandwiched between the thioredoxin gene and the receptor sequences (see FIG. 1).

Expression and Purification of the Fusion Protein Encoded in LR4:

To express the recombinant receptor-thioredoxin fusion protein, LR4 DNA was transformed into appropriate recipient expression strains. The two expression strains that we employed were AD494 trxB (DE3)pLysS (kan^{R}, Cm^{R}) and Origami trxB gor (DE3)pLysS (kan^{R}, tet^{R}, Cm^{R}). Both strains are mutant in the thioredoxin reductase locus with the Origami strain containing an additional mutation at the glutathione reductase locus. These mutations promote proper protein folding by facilitating disulfide bond formation in cytoplasmically expressed recombinant proteins in E. coli (10). Strains that were wild-type at both of these loci yielded lower levels of expression from LR4 following induction with IPTG. In addition, much of this material could not be purified in soluble form.

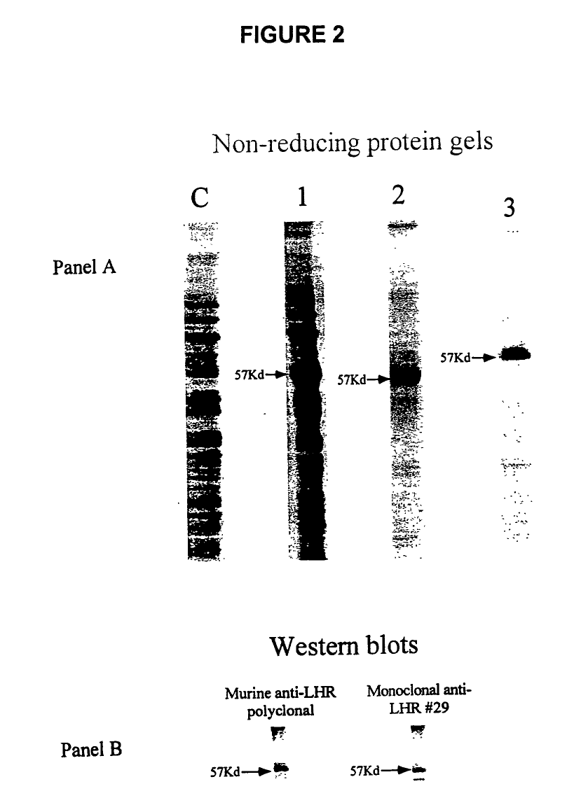

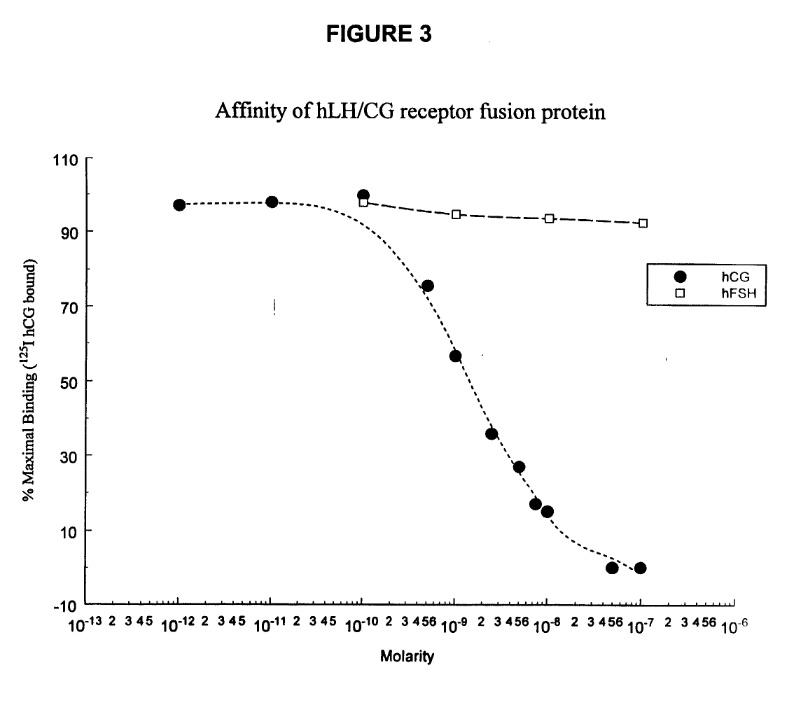

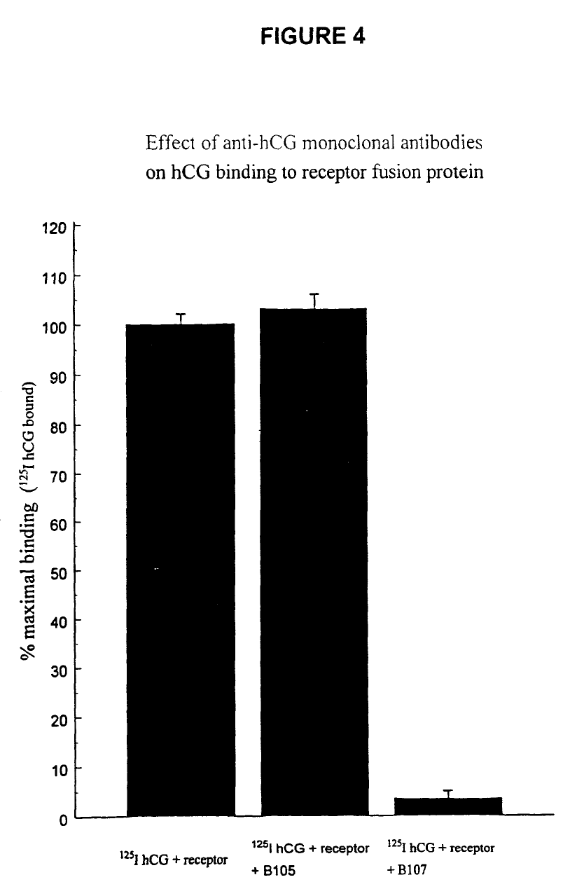

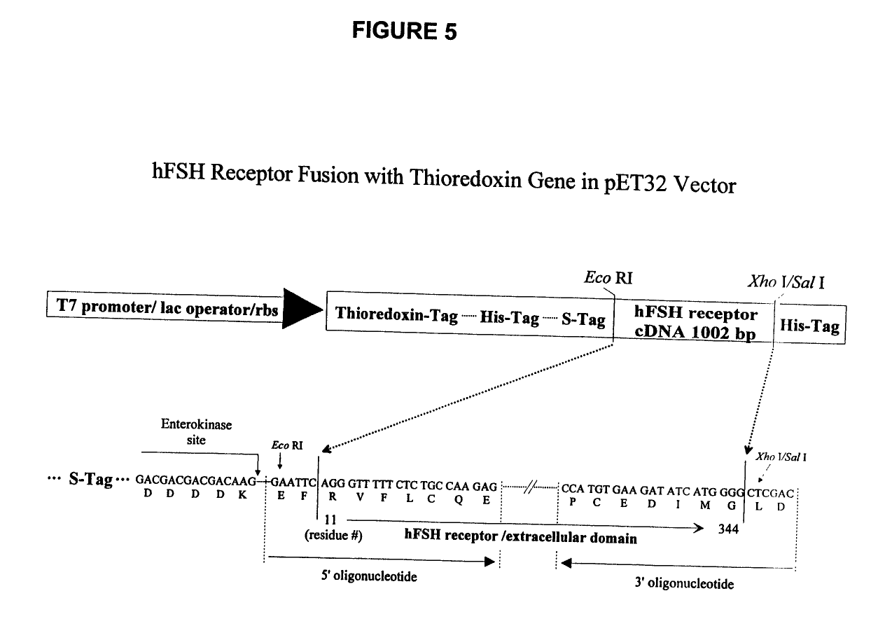

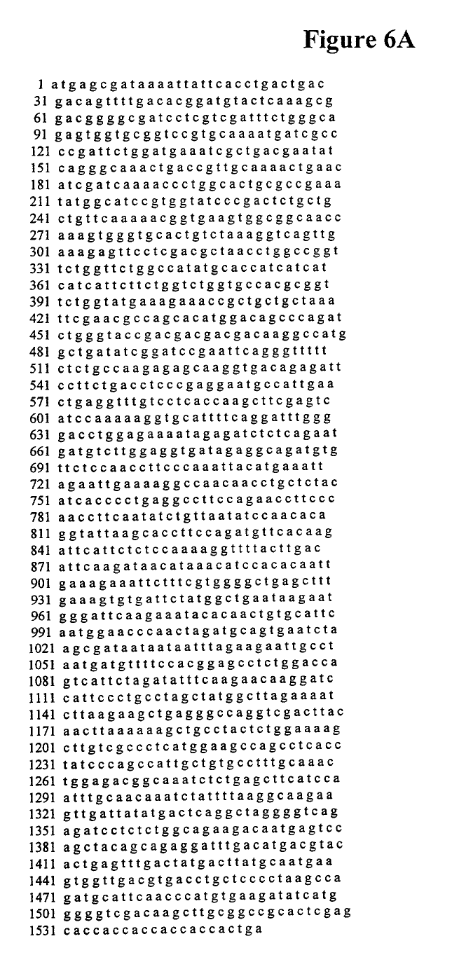

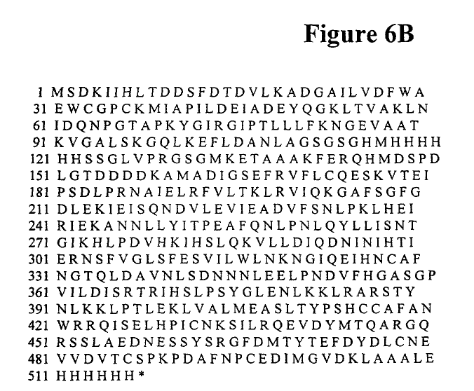

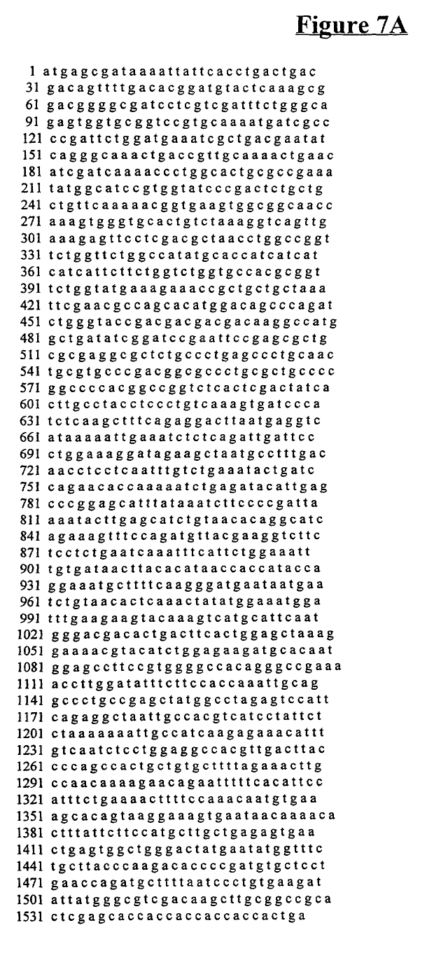

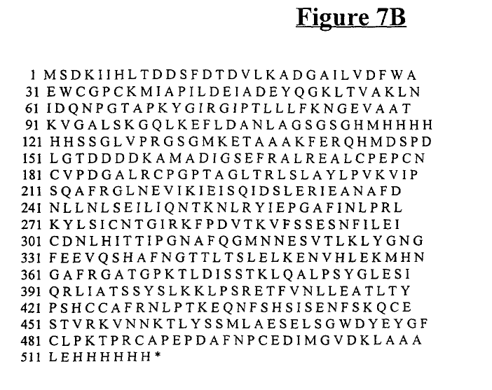

Clones from fresh transformations were picked for small overnights from which a 1:100 dilution was performed the following day for large scale expression. Cultures were grown to an optical density of 0.5 (600 nm) and then induced with IPTG at a final concentration of lmM. Induction of expression was monitored on nonreducing SDS polyacrylamide gels (see FIG. 2 panel A, lanes C and 1). Bacteria were pelleted after three hours and frozen. Comparison of expression in strains AD494 and Origami indicated that the latter strain consistently expressed higher levels of recombinant protein at each time point following induction (data not shown). As a result the Origami strain of bacteria was chosen for the expression studies.

Frozen pellets were lysed and treated with Benzonase nuclease to digest the DNA. Incubation was then continued on ice for an additional 2-3 hours or overnight at 4° C. Under these conditions the fusion protein aggregates and can be collected by centrifugation. Control pellets from an induced pET32a(+) clone in the origami strain did not yield any aggregated material following lysis and nuclease treatment. The protein aggregate was then washed with buffer twice and collected by centrifugation. Resolubilization of the protein was accomplished by resuspension of the aggregated material in buffer with the anionic detergent N-Lauroylsarcosine to a final concentration of 0.25%. The suspension was incubated at 4° C. until solubilization was complete and the solution completely clarified. At this point the recombinant protein was greater than 90% pure as determined by SDS polyacrylamide gel analysis (see FIG. 2 panel A, lane 2).

For further protein purification and to remove the detergent the solubilized material was further purified on a Ni-NTA resin column (Qiagen). The eluted fusion protein electrophoresed as a single species on a nonreducing SDS polyacrylamide gel. A sample silver stained gel is illustrated in FIG. 2 panel A, lane 3. Purified protein was soluble and could then be concentrated. Maintaining solubility of the protein at higher concentrations was facilitated by the addition of various nondetergent sulphobetaines (L. Lobel, unpublished results).

The Recombinant Receptor Protein Binds Anti-Receptor Antibodies:

To confirm that the recombinant protein expressed was the extracellular domain of the receptor, Western blot analysis was performed using polyclonal and monoclonal antibodies against the extracellular domain residues of the hLH/CG receptor. The polyclonal antisera was produced in our laboratory to a denatured polypeptide containing residues from the extracellular domain of the hLH/CG receptor that had been expressed in E. coli and was previously described (5). Monoclonal antibodies to the extracellular domain of the hLH/CG receptor, LHR29, was a gift from E. Milgrom (13). The blots are displayed in FIG. 2 panel B. They demonstrate that both polyclonal and monoclonal antibodies bound a single band that migrated at the expected molecular weight; control extracts expressing the thioredoxin protein without the receptor sequences were blank (not shown). Therefore, it was concluded that the induced fusion protein contains the extracellular domain of the hLH/CG receptor.

Affinity of the Receptor-Thioredoxin Fusion Protein for hCG:

To determine the affinity of the fusion protein for hCG a novel assay that utilized the His tag to capture receptor bound hCG was developed (see materials and methods). Briefly, purified fusion protein was incubated with ¹²⁵I-hCG and varying concentrations of cold hCG and receptor material was then separated from unbound labeled hCG with Ni-NTA resin. Resin bound material was separated by centrifugation, washed and then counted. The results of this analysis are illustrated in FIG. 3. This assay was highly reproducible and demonstrated that the affinity of the soluble receptor fusion protein for hCG was on the order of 10⁻⁹M. This is comparable to the affinity of the native receptor for hCG that was previously determined in this laboratory and by others (14-16).

To study the specificity of the fusion protein for hCG the protein was incubated with labeled hCG in varying concentrations of hFSH. As illustrated in FIG. 3 hFSH had no effect on binding at concentrations as high as 10⁻⁷M. Therefore, it was concluded that the recombinant expressed receptor domain maintains the appropriate specificity for hCG.

The Receptor-Thioredoxin Fusion Protein Binds hCG in the Proper Orientation:

To determine whether the same surface of hCG that interfaces with the native receptor also interacts with the recombinant truncated receptor, a series of experiments using epitope specific anti-hCG monoclonal antibodies that have been previously described (17,18) were performed. In particular the anti-hCG monoclonal antibodies B105 and B107 were employed. B105 can bind hCG while it is bound to the hLH/CG receptor and does not block binding of free hCG to receptor. Alternatively, B107 does not bind hCG when bound to receptor and blocks binding of free hCG. As a result the effect of these antibodies on binding of labeled hCG to the recombinant receptor using the same binding assay described above was tested. The results of this analysis are depicted in FIG. 4. They indicate that whereas B105 has little or no effect on binding of labeled hCG to the fusion protein, B107 almost completely blocks binding of the labeled hCG to the recombinant receptor. Although B105 can at times slightly enhance binding of hCG to the receptor, we have previously seen this phenomenon with native receptor on CHO cells. As a result it was concluded that hCG is bound in the proper orientation by the receptor fusion protein.

C) Discussion

The feasibility of expressing a properly folded extracellular binding domain of the hLH/CG receptor as a fusion protein in the cytoplasm of E. coli has been demonstrated. This protein can be purified in soluble form and displays a binding affinity comparable to that of native receptor. The recombinant protein binds hCG specifically and in the proper orientation. Furthermore, as we have previously shown (5), our results demonstrate that glycosylation of the binding domain of the receptor is not required for high affinity interaction with hCG.

These results are the first to demonstrate that the binding domain of the hLH/CG receptor can be expressed in the cytoplasm of E. coli and fold properly with the appropriate disulfide bonds. In addition, significant expression levels in this system were achieved. 10-30 mg/L of recombinant receptor protein were routinely expressed and this material was purified in soluble form. Although many recombinant proteins expressed cytoplasmically in E. coli are typically denatured or partially folded it was found that the reductase double mutant strain of E. coli that was utilized expressed the fusion protein as properly folded material.

The ability to express high levels of the recombinant product in an economical and expeditious system enables the design of new assays to detect novel forms of hCG or hLH. Thus far, radioimmunoassays have suggested that novel glycosylated forms of hCG are associated with Down's syndrome (19). This was based on the fortuitous identification of a unique monoclonal antibody that could detect these glycosylation differences. Nonetheless, hCG is also secreted in a variety of other pathological conditions and there may be other forms that might be distinguishable on the basis of altered receptor affinities. Implementation of a simple in vitro radioreceptor assay will therefore facilitate analysis of receptor binding affinity for hCG molecules that are secreted from a variety of malignant and benign pathologies. This could lead to a better understanding of these pathologies as well as provide new markers for diagnosis and treatment.

The availability of large quantities of a soluble form of the extracellular domain of the hLH/CG receptor also facilitates the structural characterization of a glycoprotein hormone receptor for the first time. The material produced will serve as a basis for crystallographic studies. In addition, large quantities of the extracellular domain of the natively folded receptor facilitates production of a panel of epitope specific monoclonal antibodies. Thus far, most monoclonal antibodies to the hLH/CG receptor were produced using receptor protein isolated from eukaryotic cells that express the receptor. The amount of material purified from these cell types is very small and as a result only a limited number of monoclonal antibodies have been produced thus far. Other methods for monoclonal antibody production generally employ synthetic peptides. Antibodies produced using the peptide as an immunogen are not sensitive to structural nuances of the protein and therefore are not useful for detecting conformational changes. Monoclonal antibodies generated from natively folded receptor should prove useful for studying the membrane bound structure of the receptor and examining structural variations between the unoccupied receptor and that which is docked with hCG.

The generation of human monoclonal antibodies directed against the receptor is also of interest. This is now feasible with a variety of technologies, notably that based on Abgenix's transgenic mice that express the human antibody repertoire (20). Since hLH/CG plays a prominent role in both reproductive biology and a variety of pathologies, blockade of the receptor with human monoclonal antibodies could have significant clinical application. From a reproductive standpoint, hypophyseal production of LH serves as a stimulus for testosterone production by the leydig and theca cells of the male and female gonad, respectively. Women with hirsutism and polycystic ovarian syndrome are known to have elevated levels of LH. Blockade of the hLH/CG receptor may be the most physiologic way of reducing ovarian androgen output and restoring hormonal balance in these patients. Another candidate for treatment with a receptor blocking antibody include men with androgen-dependent disorders (e.g. prostatic hypertrophy and carcinoma).

In both sexes, activation of the hLH/CG receptor is crucial for gametogenesis: in women to induce final oocyte maturation and ovulation, and in men to facilitate spermatogenesis (via the generation of testosterone). A fertility vaccine based on the carboxyterminal peptide (CTP) of beta-hCG (21), and another against the entire beta subunit (22) have undergone clinical trials in women. Feasibility of a receptor based vaccine has been demonstrated in rodents by the inhibition of gonadotropin action with soluble extracellular domain of the FSH receptor that was produced in a eukaryotic expression system (9). Preliminary evidence in primates also supports the efficacy of an LH receptor vaccine in control of fertility (23,24). The potential for conjugating the soluble human receptor to an immunogenic compound for use in a contraceptive vaccine is therefore feasible. Furthermore, anti-receptor antibodies might offer a more effective blockade of ligand binding than anti-hCG antibodies since the immune system doesn't have to overcome a large circulating reservoir of antigen but instead has to occupy a relatively more stable level of cell surface bound receptor. Alternatively, another strategy for contraception is passive immunization with a human anti-receptor monoclonal antibody. Depot formulations could circumvent any reliance on an adequate immune response for contraception, and have a finite duration of effect. Women who cannot tolerate estrogens and progestins, or in whom hormonal forms of contraception are contraindicated may benefit from these novel contraceptive methods.

Anti-receptor antibodies might also be used for emergency contraception or as an abortifacient. Antibodies to hCG have been shown to prevent implantation of marmoset embryos (25,26) and induce pregnancy loss in baboons (27). Inhibitors of the hCG receptor should yield comparable results and represents a potential alternative to hormonal methods of postcoital contraception.

Aside from the placental trophoblast, a number of neoplasias are known to produce hCG. Structural studies have identified it as a member of the family of cysteine knot growth factors (28) and in vitro studies suggests that it plays a role as a promotor of cell proliferation (29). In addition, there has been speculation that hCG confers immune tolerance and/or resistance to apoptosis (30,31); properties similarly ascribed to the embryonic trophoblast. For example, addition of hCG to culture media of an epithelial ovarian cancer cell line suppressed cisplatin-induced apoptosis (32). Expression in vivo has also been shown to be associated with a more advanced stage of disease and resistance to chemotherapy in patients with hCG-producing lung cancers (33). Therefore, its presence may not merely be a by-product of oncogenesis and embryonic gene activation, but rather an essential effector of the neoplastic process.

Although a clear role for the receptor in neoplasia has not been established studies have indicated that antibodies to hCG and its subunits inhibit growth of lung cancer cells in vitro (22). Furthermore, vaccines directed against β-hCG have demonstrated efficacy for treating a variety of tumors in animal models, and clinical trials using human anti-hCG monoclonal antibodies are currently ongoing in patients with colorectal, pancreatic and prostate cancer (Avicine, AVI Biopharma, Portland, Oreg.). The use of receptor blocking antibodies in anti-cancer therapy has been validated by the approval of Herceptin, a humanized monoclonal antibody directed against the surface growth factor her2-NEU, as treatment for metastatic breast carcinoma. Blockade of the hCG receptor may have similar applicability as a chemotherapeutic and/or chemopreventive agent.

In summary, the ability to efficiently produce the extracellular domain of the hLH/CG receptor in a pure, soluble form facilitates basic work on receptor structure and may have significant clinical application. Ultimately, elucidation of the quaternary structure of the ligand binding domain of the receptor will facilitate the development of small molecule antagonists and agonists of the receptor.

FSH Receptor

Construction of an Expression Clone Containing the Extracellular Domain of the hFSH Receptor:

To express the extracellular domain of the hFSH receptor in E. coli, a cDNA encoding the extracellular domain of the hLH/CG receptor was fused with a number of different proteins that were known to enhance stability and promote folding of the tethered sequences. One clone, designated FR8, expressed significant amounts of material upon induction. It contained a fusion of the receptor sequences downstream of a thioredoxin gene in a pET32a(+) vector and is the source of all the recombinant material described in this paper. Clone FR8 contains residues 11-334 from the mature receptor protein. These residues encompass nearly the entire extracellular domain of the receptor.

Expression and Purification of the Fusion Protein Encoded in FR8:

To express the recombinant receptor-thioredoxin fusion protein, FR8 DNA was transformed into appropriate recipient expression strains. The two expression strains that we employed were AD494 trxB (DE3)pLysS (kan^{R}, Cm^{R}) and Origami trxb gor (DE3)pLysS (kan^{R}, tet^{R}, Cm^{R}). Both strains are mutant in the thioredoxin reductase locus with the Origami strain containing an additional mutation at the glutathione reductase locus. These mutations promote proper protein folding by facilitating disulfide bond formation in cytoplasmically expressed recombinant proteins in E. coli (48). Strains that were wild-type at both of these loci yielded lower levels of expression from FR8 following induction with IPTG.

The induction of expression on non-reducing SDS polyacrylamide gels were routinely monitored. Comparison of expression in strains AD494 and Origami indicated that the latter strain consistently expressed higher levels of recombinant protein at each time point following induction (data not shown). As a result, the Origami strain of bacteria for all expression studies was chosen.

The induced fusion protein was initially harvested as aggregated material which was resolubilized in lOmM phosphate buffer pH 8.0 without any salt or detergent. In general the aggregated protein was able to be resolubilized in this buffer by incubating the suspension at 37° C. for 1 hour. Occasionally, N-Lauroylsarcosine was added to a concentration of 0.2% to facilitate solubilization. This material was greater than 90% pure as determined by SDS polyacrylamide gel analysis. Further protein purification of the fusion protein was accomplished by Ni-NTA resin affinity chromatography (Qiagen). The eluted fusion protein electrophoresed as a single species. Purified protein was soluble and could then be concentrated. Maintaining solubility of the protein at higher concentrations was facilitated by the addition of various nondetergent sulphobetaines.

Affinity of the Receptor-Thioredoxin Fusion Protein for hFSH:

To determine the affinity of the fusion protein for hFSH we developed a novel assay that utilized the His tag to capture receptor bound hFSH (see materials and methods). Briefly, purified fusion protein was incubated with ¹²⁵I-hFSH and varying concentrations of cold hFSH and receptor material was then separated from unbound labeled hFSH with Ni-NTA resin. Resin bound material was separated by centrifugation, washed and then counted. This assay was highly reproducible and demonstrated that the affinity of the soluble receptor fusion protein for hFSH was on the order of 10⁻⁹M. This is comparable to the affinity of the native receptor for hFSH.

To study the specificity of the fusion protein for hFSH the protein was incubated with labeled hFSH in varying concentrations of hCG. The hCG had no effect on binding at concentrations as high as 10⁻⁷M. Therefore, it was concluded that the recombinant expressed receptor domain maintains the appropriate specificity for hFSH.

Bacterial Strains:

All molecular biology techniques and large scale preparation of plasmid DNA was performed with E. coli strain DH5α. Expression constructs were transformed into either strain AD494 trxB (DE3)pLysS (kan^{R}, Cm^{R}) or Origami trxB gor (DE3)pLysS (kan^{R}, tet^{R}, Cm^{R}). Both strains were acquired from Novagen (Madison, Wis.) and are mutant at the thioredoxin reductase locus (trxB). The Origami strain is also mutant at the glutathione reductase locus and is designated gor. For expression of fusion constructs the strains were freshly transformed and colonies were picked directly from the transformation plates into growth media for expression. Passing or freezing transformants leads to diminished expression from the bacterial population. Strains were grown in standard Luria-Bertani medium (LB) for expression. Richer media formulations such as SOC media lead to poorer expression yields of soluble protein.

Gonadotropin Preparations:

The preparations of hCG (urinary hCG CR127) used in this study have previously been described (49,50). The CR127 preparation of hCG is the widely distributed reference preparation and was generously provided by Dr. Steven Birken (50). Human FSH was acquired from the National Pituitary Agency (NIH).

Molecular Biology:

All enzymes for recombinant DNA were purchased from New England Biolabs. DNA primers for PCR were synthesized by the Columbia University Core Laboratory. The 5′ primer introduced an Eco RI site in the same frame and adjacent to the receptor sequence whereas the 3′ primer introduced an in frame Xho I site. PCR reactions were performed with Vent DNA polymerase (New England Biolabs) and all products of the reactions were sequenced to ensure that no mutations were introduced during the amplification process. Ligation reactions were transformed into DH5α and DNA clones were grown in DH5 for large scale plasmid preparation. Transformation of DH5α and the expression strains AD494 and Origami were performed according to standard techniques with calcium chloride.

Construction of Thioredoxin Fusion Clone:

The expression vector for these experiments was pET32a(+) (Novagen, Madison Wis.). DNA encoding the extracellular domain of the hLH/CG receptor was isolated by PCR of a full length clone of the hFSH receptor (kindly provided by Dr. Aaron Hsueh). Using primers with engineered 5′ Eco RI and 3′ Xho I sites, a cDNA encoding residues 11-334 of the receptor was amplified by PCR. The amplified product contained in frame 5′ Eco RI and 3′ Xho I sites. This was inserted into the polylinker cloning site of the pET32a(+) vector from Novagen. The resulting construct has the receptor sequences fused downstream of the thioredoxin gene. In addition, the insertion generates a frameshift such that the 3′ residues at the carboxy terminus encoded in this clone are equivalent to those encoded by the pET32b(+) clone. These terminal residues in pET32b(+) comprise a second His tag. Therefore this fusion clone encodes a second His tag at the 3′ end with the other tag sandwiched between the thioredoxin gene and the receptor sequences. The clone utilized for all expression studies was designated FR8.

Expression of Fusion Protein:

For expression in AD494, colonies were inoculated into 10 mL of LB media with kanamycin (50 μg/mL) and ampicillin (75 μg/mL) and grown overnight. The fresh overnight culture was then diluted 1:100 into LB with 50 μg/mL ampicillin and grown to an OD of 0.5 at 600 nm. The culture was then induced with 1 mM isopropyl-thio-?-D-thiogalactoside (IPTG) for 3 hours and the bacteria were harvested by centrifugation. Induction of expression was monitored on nonreducing SDS polyacrylamide gels. Bacterial pellets were then frozen and stored at −20° C. Expression in the Origami strain was essentially the same except that the overnight culture was grown in the presence of tetracycline (25 μg/mL) in addition to ampicillin and kanamycin.

Purification of Expressed Fusion Protein:

Frozen bacterial pellets were resuspended in 1 mL of Bugbuster reagent (Novagen, Madison Wis.) per 50 mL of bacterial culture with the addition of 20 μg/mL of lysozyme. The suspended cells were allowed to sit on ice until lysis was complete and the suspension became viscous. The lysed cells were then incubated with 25 units of Benzonase nuclease (Novagen, Madison Wis.) per mL. The mixture was incubated on ice until the viscosity disappeared. Aggregated protein was collected by further incubation of the material on ice for either 2-3 hours or overnight at 4° C. followed by centrifugation. Control pellets from an induced pET32a(+) clone in the origami strain did not yield any aggregated material following lysis and nuclease treatment. The protein aggregate was then washed with buffer twice and collected by centrifugation. The protein pellet was resuspended in 10 mM NaHPO₄ pH 8.0 at 1 mL/mL of suspended cells. The material was mixed well by pipetting up and down through the tip of an eppendorf pipette to help disperse the aggregated protein. Occasionally, N-Lauroylsarcosine was added to a concentration of 0.2% to facilitate solubiliztion. The resuspended protein was then allowed to sit at 37° C. until the solution clarified and solubilization was complete. If solubilization was not complete the solution was diluted twofold. Incomplete initial solubilization generally indicated that bacterial expression exceeded 10 mg/L and twofold dilution of the solution led to complete solubilization in all cases. In general, detergent was not required to solubilize the hFSH receptor fusion protein as was the case for the hLH/CG receptor fusion. At this point the recombinant protein was greater than 90% pure as determined by SDS polyacrylamide gel analysis.

For further protein purification the solubilized material was purified on a Ni-NTA resin column (Qiagen). Solubilized fusion protein was diluted 10 fold in 50 mM NaHPO₄/300 mM NaCl pH 8.0 with the addition of N-Lauroylsarcosine to a final concentration of 0.05%. This material was passed through a Ni-NTA resin column (Qiagen) and the flow through was recirculated through the resin 5 times. The resin was then washed with five volumes of 50 mM NaHPO₄/300 mM NaCl pH 8.0 followed by the same buffer with the addition of 10 or 20 mM Imidiazole pH 8.0 for the second and third washes respectively. The bound fusion protein was then eluted with three column volumes of 50 mM NaHPO₄/300 mM NaCl/250 mM Imidazole pH 8.0. The eluted material was then exhaustively dialyzed in 10 mM NaHPO₄ pH 8.0.

The eluted fusion protein electrophoresed as a single species on a nonreducing SDS polyacrylamide gel. Purified protein was soluble and could be concentrated. Maintaining solubility of the protein at high concentrations was facilitated by the addition of various nondetergent sulphobetaines.

Electrophoresis:

SDS-polyacrylamide gel electrophoresis was performed according to the procedure described by Laemmli (51,52). The sample buffer contained 125 mM Tris-HCl, pH 6.8, 10% glycerol, 2%SDS, 0.01% bromophenol blue. Gels were 10% polyacrylamide and either coomassie blue or silver stained according to established techniques (53).

Western Blot Analysis:

Following electrophoresis, the proteins in the gel were transferred to nitrocellulose paper using a variation of the methods of Towbin (54) and Burnette (55). After blocking in 5% BSA, 0.01M Tris-HCl, 0.15M NaCl pH 7.6 (BSA-TBS), the paper was incubated overnight with antibody diluted in BSA-TBS (at concentrations of approximately 3-4 μg antibody/ml for monoclonal antibodies and using a dilution of 1:500 of anti-hFSH receptor antiserum for polyclonal antibodies). The binding of the primary antibodies was visualized as previously described (56).

Determination of the Affinity of the Fusion Protein for hFSH:

To determine the affinity of the fusion protein for hFSH we developed a reliable and highly reproducible technique for assaying binding using the His tag for capture of receptor bound material. This was accomplished by using the Ni-NTA resin to capture receptor fusion protein following incubation with radiolabeled ¹²⁵I-hFSH. Briefly, purified receptor material was incubated in 50 mM NaHPO₄/300 mM NaCl pH 8.0 with ¹²⁵I-hFSH for 2-4 hours at ambient temperature or at 4° C. overnight. Incubation volume was standardized at 1 mL and approximately 200 ng of receptor fusion protein with 2×10⁵ cpm of labeled hFSH (approximately 75 fm) was added to each incubation reaction. Ni-NTA resin was prepared by extensive washing with SOmM NaHPO₄/300 mM NaCl pH 8.0 followed by resuspension in the same buffer with a volume equivalent to packed resin volume. Typically 20 μL of the resuspended resin was added to each incubation reaction and the suspension was incubated an additional 2 hours at ambient temperature with mixing every 10 minutes to resuspend the settled resin. The resin was then spun down and washed twice with 50 mM NaHPO₄/300 mM NaCl pH 8.0 followed by the same buffer including 20 mM Imidazole at pH 8.0. Bound hFSH was then identified by counting in a gamma counter. Background generated by binding of the labeled hFSH alone to the resin was generally less than 10% of that obtained when receptor material was added to the incubation.

To facilitate an accurate determination of affinity, 2×10⁵ cpm of ¹²⁵I-hFSH was titrated with decreasing amounts of receptor material until the number of bound counts began to decrease. At that point the amount of receptor material was not in excess and was just sufficient to bind the maximum number of counts (i.e., as many counts as a saturating amount of receptor material). Using this same amount of receptor material (approximately 200 ng) with 2×10⁵ cpm of ¹²⁵I-hFSH (approximately 75 fm) in each incubation reaction the affinity of the expressed receptor fusion for hFSH was determined by adding decreasing amounts of cold hFSH to each incubation reaction such that the concentration of cold hFSH varied between 10⁻⁷ and 10⁻¹²M. The data was plotted as molarity of cold hFSH versus % maximally bound ¹²⁵I-hFSH. Maximally bound ¹²⁵I-hFSH is equal to the number of counts bound in the absence of cold hFSH. The % maximally bound ¹²⁵I-hFSH is then determined from the equation [cpm bound hFSH in presence of test amount of cold hFSH]/[cpm maximally bound hFSH]×100. The % maximally bound ¹²⁵I-hFSH is presented instead of absolute counts so that data could be readily compared from a series of binding experiments that may have employed different preparations of labeled hFSH and receptor protein. Graphs were plotted with the program Axum 5.0 (Mathsoft, Cambridge, Mass.) and a best fit curve was drawn through the data points using the Lowess algorithm.

Discussion:

Using a technique similar to that described previously for expressing and purifying the extracellular domain of the hCG receptor, high levels of expression of the extracellular domain of the hFSH receptor in the cytoplasm of E. Coli have been achieved. Expression of the receptor as a fusion protein with thioredoxin facilitates disulfide bond formation and proper folding of the protein. The recombinant receptor fragment binds hFSH specifically, and is purified and isolated in soluble form.

Native glycoprotein hormone receptors are glycosylated, although little is known about the functional role of these carbohydrate moieties. The extracellular domain of the hFSH receptor that were expressed in bacteria is not post-translationaly glycosylated yet hFSH binds with high affinity and specificity. This suggests that glycosylation of these residues, while perhaps functionally necessary for proper activation of the secondary messenger (cAMP) cascade, plays a negligible role in hormone-receptor binding.

The ability to generate large quantities of the recombinant receptor facilitates attempts at crystallizing and elucidating the structure of the binding domain of the hFSH receptor. Isolation of purified receptor can also be used to generate a panel of monoclonal antibodies. These are used to further study the membrane bound structure of the receptor and elucidate specific epitopes which mediate hormone binding. Determining the quarternary structure of the extracellular domain of the receptor enables production of small molecule agonists and antagonists with potential clinical applicability.

HFSH, a member of the glycoprotein hormone family, is the primary stimulus for ovarian follicular development in women and a facilitator of spermatogenesis in men. An hFSH vaccine has been shown to reduce sperm counts in men, but not to contraceptive levels. A similar vaccine using the hFSH receptor as an immunogen may achieve superior results. Active or passive immunization to the receptor should also reduce ovarian estrogen production. This may be desirable for women with estrogen-dependant pathologies, including endometriosis, fibroids, and some malignancies (e.g. breast cancer).

The ability to efficiently produce the extracellular domain of the FSH receptor in a pure, soluble form will thus facilitate basic work on receptor structure and may have significant clinical application. This represents our second successful attempt at recombinant expression of a properly folded glycoprotein receptor fragment, validating our bacterial expression system.

FSH testing is an integral part of the fertility evaluation in women of advanced reproductive age. Current testing is not standardized, and significant interassay variation exists. A receptor-based binding assay may be more accurate (i.e. less variable) and cheaper than antibody-based assays (for the ligand) currently in use.

Physiologically, FSH is required for follicular development. As a vaccine or through the generation of anti-receptor antibodies, receptor inhibition prevents formation of a dominant follicle and ovulation, offering the a use as a female contraceptive,

The requirement for FSH for spermatogenesis in men is not absolute. Ovine FSH, when administered to bonnet monkeys, induced oligospermia (not azospermia). Phase I clinical trials of an FSH-based vaccine in humans has been completed. Sperm counts diminished, but not to the extent expected. A similar vaccine using the FSH receptor as an immunogen may achieve better results.

Inducing a low estrogen state may be beneficial for women with estrogen-dependant pathology. This includes women with endometriosis and uterine fibroids, as well as malignancies (e.g. breast cancer). GnRH agonists, which are used clinically to induce a hypogonadotropic state, are approved for use in premenopausal patients with endometriosis and fibroids. Tamoxifen, an estrogen antagonist at the level of the breast, is approved for treatment and prevention of estrogen receptive positive breast cancer. FSH plays the key role in ovarian follicogenesis and subsequent estrogen production. FSH receptor antibodies capable of blocking the receptor may have similar potential in these conditions.

### REFERENCES

1. Ingley, E. and Hemmings, B. A. (1999). Large-scale expression and purification of a soluble form of the pleckstrin homology domain of the human protooncogenic serine/threonine protein kinase PKB (c-akt) in Escherichia coli. Protein Expr.Purif. 17, 224-230.

2. Qiu, J., Swartz, J. R., and Georgiou, G. (1998). Expression of active human tissue-type plasminogen activator in Escherichia coli. Appl.Environ.Microbiol. 64, 4891-4896.

3. Cheadle, C., Ivashchenko, Y., South, V., Searfoss, G. H., French, S., Howk, R., Ricca, G. A., and Jaye, M. (1994). Identification of a src sh3 domain binding motif by screening a random phage display library. J.Biol.Chem. 269, 24034-24039.

4. Huth, J. R., Norton, S. E., Lockridge, O., Shikone, T., Hsueh, A. J., and Ruddon, R. W. (1994). Bacterial expression and in vitro folding of the beta-subunit of human chorionic gonadotropin (hCG beta) and functional assembly of recombinant hCG beta with hCG alpha. Endocrinology 135, 911-918.

5. Lobel, L. I., Rausch, P., Trakht, I., Pollak, S., and Lustbader, J. W. (1997). Filamentous phage displaying the extracellular domain of the hLH/CG receptor bind hCG specifically. Endocrinology 138, 1232-1239.

6. di Marzo, V., Willis, A. E., Boyer-Thompson, C., Appella, E., Perham, and RN (1994). Structural mimicry and enhanced immunogenicity of peptide epitopes displayed on filamentous bacteriophage. The V3 loop of HIV-1 gp120. J.Mol.Biol. 243, 167-172.

7. Greenwood, J., Willis, A. E., and Perham, R. N. (1991). Multiple display of foreign peptides on a filamentous bacteriophage. Peptides from Plasmodium falciparum circumsporozoite protein as antigens. J.Mol.Biol. 220, 821-827.

8. Chen, W. and Bahl, O. P. (1993). High expression of the hormone binding active extracellular domain (1-294) of rat lutropin receptor in Escherichia coli. Molecular.&.Cellular.Endocrinology 91, 35-41.

9. Osuga, Y., Kudo, M., Kaipia, A., Kobilka, B., and Hsueh, A. J. (1997). Derivation of functional antagonists using N-terminal extracellular domain of gonadotropin and thyrotropin receptors. Mol.Endocrinol. 11, 1659-1668.

10. Prinz, W. A., Aslund, F., Holmgren, A., and Beckwith, J. (1997). The role of the thioredoxin and glutaredoxin pathways in reducing protein disulfide bonds in the Escherichia coli cytoplasm. J.Biol.Chem. 20;272, 15661-15667.

11. Stewart, E. J., Aslund, F., and Beckwith, J. (1998). Disulfide bond formation in the Escherichia coli cytoplasm: an in vivo role reversal for the thioredoxins. EMBO J. 17, 5543-5550.

12. Wimalasena, J., Dostal, R., and Meehan, D. (1992). Gonadotropins, estradiol, and growth factors regulate epithelial ovarian cancer cell growth. Gynecologic Oncology 92, 345-350.

13. Meduri, G., Charnaux, N., Loosfelt, H., Jolivet, A., Spyratos, F., Brailly, S., and Milgrom, E. (1997). Luteinizing hormone/human chorionic gonadotropin receptors in breast cancer. Cancer Res. 57, 857-864.

14. Lobel, L., Pollak, S., Wang, S., Chaney, M., and Lustbader, J. W. (1999). Expression and characterization of recombinant beta-subunit hCG homodimer. Endocrine. 10, 261-270.

15. Dufau, M. L. and Kusuda, S. (1987). Purification and characterization of ovarian LH/hCG and prolactin receptors. Journal.of.Receptor.Research. 7, 167-193.

16. Dufau, M. L. (1998). The luteinizing hormone receptor. Annu Rev Physiol 60, 461-496.

17. Lustbader, J. W., Yarmush, D. L., Birken, S., Puett, D., and Canfield, R. E. (1993). The application of chemical studies of human chorionic gonadotropin to visualize its three-dimensional structure. [Review]. Endocrine.Reviews. 14, 291-311.

18. O'Connor, J. F., Birken, S., Lustbader, J. W., Krichevsky, A., Chen, Y., and Canfield, R. E. (1994). Recent advances in the chemistry and immunochemistry of human chorionic gonadotropin: impact on clinical measurements. [Review]. Endocrine.Reviews. 15, 650-683.

19. Cole, L. A., Shahabi, S., Oz, U. A., Bahado-Singh, R. O., and Mahoney, M. J. (1999). Hyperglycosylated human chorionic gonadotropin (invasive trophoblast antigen) immunoassay: A new basis for gestational Down syndrome screening. Clin.Chem. 45, 2109-2119.

20. Green, L. L. (1999). Antibody engineering via genetic engineering of the mouse: XenoMouse strains are a vehicle for the facile generation of therapeutic human monoclonal antibodies. J.Immunol.Methods 231, 11-23.

21. Jones, W. R., Bradley, J., Judd, S. J., Denholm, E. H., Ing, R. M., Mueller, U. W., Powell, J., Griffin, P. D., and Stevens, V. C. (1988). Phase I clinical trial of a World Health Organisation birth control vaccine. Lancet 1, 1295-1298.

22. Talwar, G. P. (1999). Vaccines and passive immunological approaches for the control of fertility and hormone-dependent cancers. Immunol.Rev. 171, 173-192.

23. Pal, R., Talwar, G. P., Saxena, A., Sam, M. G., Rathnam, P., and Saxena, B. B. (1992). Biological actions of monoclonal antibodies to bovine lutropin receptor. J.Reprod.Immunol. 22, 87-103.

24. Pal, R., Singh, O. M., Talwar, G. P., Singh, M., and Saxena, B. B. (1992). Active immunization of baboons (Papio anubis) with the bovine LH receptor. J.Reprod.Immunol. 21, 163-174.

25. Hearn, J. P. (1986). The embryo-maternal dialogue during early pregnancy in primates. J.Reprod.Fertil. 76, 809-819.

26. Hearn, J. P., Webley, G. E., and Gidley-Baird, A. A. (1991). Chorionic gonadotrophin and embryo-maternal recognition during the peri-implantation period in primates. J.Reprod.Fertil. 92, 497-509.

27. Tandon, A., Das, C., Jailkhani, B. L., and Talwar, G. P. (1981). Efficacy of antibodies generated by Pr-beta-hCG-TT to terminate pregnancy in baboons: its reversibility and rescue by medroxyprogesterone acetate. Contraception 24, 83-95.

28. Wu, H., Lustbader, J. W., Liu, Y., Canfield, R. E., and Hendrickson, W. A. (1994). Structure of human chorionic gonadotropin at 2.6 A resolution from MAD analysis of the selenomethionyl protein. Structure. 2, 545-558.

29. Melmed, S. and Braunstein, G. D. (1983). Human chorionic gonadotropin stimulates proliferation of Nb 2 rat lymphoma cells. J.Clin.Endocrinol.Metab 56, 1068-1070.

30. Regelson, W. (1995). Have we found the “definitive cancer biomarker”? The diagnostic and therapeutic implications of human chorionic gonadotropin-beta expression as a key to malignancy [editorial; comment]. Cancer 76, 1299-1301.

31. Triozzi, P. L. and Stevens, V. C. (1999). Human chorionic gonadotropin as a target for cancer vaccines. Oncol.Rep. 6, 7-17.

32. Kuroda, H., Mandai, M., Konishi, I., Yura, Y., Tsuruta, Y., Hamid, A. A., Nanbu, K., Matsushita, K., and Mori, T. (1998). Human chorionic gonadotropin (hCG) inhibits cisplatin-induced apoptosis in ovarian cancer cells: possible role of up-regulation of insulin-like growth factor-1 by hCG. Int.J.Cancer 76, 571-578.

33. Szturmowicz, M., Slodkowska, J., Zych, J., Rudzinski, P., Sakowicz, A., and Rowinska-Zakrzewska, E. (1999). Frequency and clinical significance of beta-subunit human chorionic gonadotropin expression in non-small cell lung cancer patients. Tumour.Biol. 20, 99-104.

34. Birken, S., Chen, Y., Gawinowicz, M. A., Lustbader, J. W., Pollak, S., Agosto, G., Buck, R., and O'Connor, J. (1993). Separation of nicked human chorionic gonadotropin (hCG), intact hCG, and hCG beta fragment from standard reference preparations and raw urine samples. Endocrinology 133, 1390-1397.

35. Birken, S., Gawinowicz, M. A., Kardana, A., and Cole, L. A. (1991) The heterogeneity of human chorionic gonadotropin (hCG). II. Characteristics and origins of nicks in hCG reference standards. Endocrinology 129, 1551-1558.

36. Laemmli, U. K. and Favre, M. (1973). Maturation of the head of bacteriophage T4. I. DNA packaging events. J.Mol.Biol. 73, 575-599.

37. Laemmli, U. K. (1970). Cleavage of structural proteins during the assembly of the head of bacteriophage T4. Nature 70, 680-685.

38. Morrissey, J. H. (1981). Silver stain for proteins in polyacrylamide gels: a modified procedure with enhanced uniform sensitivity. Anal.Biochem. 117, 307-310.

39. Towbin, H., Staehelin, T., and Gordon, J. (1979). Electrophoretic transfer of proteins from polyacrylamide gels to nitrocellulose sheets: procedure and some applications. Proc.Natl.Acad.Sci.U.S.A. 76, 4350-4354.

40. Burnette, W. N. (1981). “Western blotting”: electrophoretic transfer of proteins from sodium dodecyl sulfate—polyacrylamide gels to unmodified nitrocellulose and radiographic detection with antibody and radioiodinated protein A. Anal.Biochem. 112, 195-203.

41. Acevedo, H. F. et al. (1995) “Human chorionic gonadotropin-beta subunit gene expression in cultured human fetal and cancer cells of different types and origins” Cancer 76: 1467-1475.

42. Melmed, S. et al. (1983) “Human chorionic gonadotropin stimulates proliferation of Nb 2 rat lymphoma cells” J. Clin. Endocrinol. Metab 56: 1068-1070.

43. Bhalang, K. et al. (1999) “Immunohistochemical study of the expression of human chorionic gonadotropin-beta in oral squamous cell carcinoma” Cancer 85:757-762.

44. Szturmowicz, N. et al. (1999) “Frequency and clinical significance of beta-subunit human chorionic gonadotropin expression in non-small cell lung cancer patients” Tumour Biol 20:99-104.

45. Hedstrom, J. et al. (1999) “Concentration of free hCGbeta subunit in serum as a prognostic marker for squamous-cell carcinoma of the oral cavity and oropharynx” Int. J. Cancer 84:525-528.

46. Talwar, G. P. (1999) “Vaccines and passive immunological approaches for the control of fertility and hormone-dependent cancers” Immunol.Rev. 171:173-192.

47. Kalantarov, G. et al. (1998) Demonstration of dose dependent cytotoxic activity in cancer cells by specific human chorionic gonadotropin monoclonal antibodies. Cancer 83:783-787.

48. Prinz, W. A., F. Aslund, A. Holmgren, and J. Beckwith. 1997. The role of the thioredoxin and glutaredoxin pathways in reducing protein disulfide bonds in the Escherichia coli cytoplasm. J. Biol.Chem. %20;272:15661-15667.

49. Birken, S., Y. Chen, M. A. Gawinowicz, J. W. Lustbader, S. Pollak, G. Agosto, R. Buck, and J. O'Connor. 1993. Separation of nicked human chorionic gonadotropin (hCG), intact hCG, and hCG beta fragment from standard reference preparations and raw urine samples. Endocrinology 133:1390-1397.

50. Birken,S., M. A. Gawinowicz, A. Kardana, and L. A. Cole. 1991. The heterogeneity of human chorionic gonadotropin (hCG). II. Characteristics and origins of nicks in hCG reference standards. Endocrinology 129:1551-1558.

51. Laemmli, U. K. and M. Favre. 1973. Maturation of the head of bacteriophage T4. I. DNA packaging events. J.Mol.Biol. 73:575-599.

52. Laemmli, U. K. 1970. Cleavage of structural proteins during the assembly of the head of bacteriophage T4. Nature 70:680-685.

53. Morrissey, J. H. 1981. Silver stain for proteins in polyacrylamide gels: a modified procedure with enhanced uniform sensitivity. Anal.Biochem. 117:307-310.

54. Towbin, H., T. Staehelin, and J. Gordon. 1979. Electrophoretic transfer of proteins from polyacrylamide gels to nitrocellulose sheets: procedure and some applications. Proc.Natl.Acad.Sci.U.S.A. 76:4350-4354.

55. Burnette, W. N. 1981. “Western blotting”: electrophoretic transfer of proteins from sodium dodecyl sulfate—polyacrylamide gels to unmodified nitrocellulose and radiographic detection with antibody and radioiodinated protein A. Anal.Biochem. 112:195-203.

56. Lustbader, J. W., D. L. Yarmush, S. Birken, D. Puett, and R. E. Canfield. 1993. The application of chemical studies of human chorionic gonadotropin to visualize its three-dimensional structure. [Review]. Endocrine.Reviews. 14:291-311.

57. U.S. Pat. No. 4,816,567, issued Mar. 28, 1989 to Cabilly et al.

58. U.S. Pat. No. 5,225,539, issued Jul. 6, 1993 to Gregory Winter.

59. U.S. Pat. No. 5,585,089, issued Dec. 17, 1996 to Queen et al.

60. U.S. Pat. No. 5,693,761, issued Dec. 2, 1997 to Queen et al.

61. PCT International Application No. PCT/US89/05857, filed Dec. 28, 1989, published Jul. 26, 1990, WO 90/07861.

What is claimed:

1. A nucleic acid which encodes a soluble polypeptide which comprises an extracellular domain of a gonadotropin receptor and thioredoxin, wherein the soluble polypeptide is capable of binding to the gonadotropin.

2. A nucleic acid which encodes a soluble polypeptide which comprises an extracellular domain of a gonadotropin receptor and a peptide segment comprising consecutive histidine residues, wherein the soluble polypeptide is capable of binding to the gonadotropin.

3. The nucleic acid of claim 1, wherein the polypeptide further comprises a peptide segment comprising consecutive histidine residues.

4. The nucleic acid of any one of claims 1-3, wherein the gonadotropin receptor is a human luteinizing hormone/choriogonadotropin receptor and the soluble polypeptide is capable of binding to human luteinizing hormone or human chorionic gonadotropin.

5. The nucleic acid of claim 4, wherein the extracellular domain of human luteinizing hormone/choriogonadotropin receptor comprises consecutive amino acids having the sequence set forth in SEQ ID NO:2 from the R at position 168 to the G at position 509.

6. The nucleic acid of claim 4, wherein the thioredoxin comprises consecutive amino acids having the sequence set forth in SEQ ID NO:2 from the M at position 1 to the A at position 109.

7. The nucleic acid of any one of claims 1-3, wherein gonadotropin receptor is a human follicle stimulating hormone receptor and the soluble polypeptide is capable of binding to human follicle stimulating hormone.

8. The nucleic acid of claim 7, wherein the extracellular domain comprises consecutive amino acids having the sequence set forth in SEQ ID NO:4 from the R at position 168 to the G at position 501.

9. The nucleic acid of claim 7, wherein the thioredoxin comprises consecutive amino acids having the sequence set forth in SEQ ID NO:4 from the M at position 1 to the A at position 109.

10. The nucleic acid of claim 2 or 3, wherein the peptide segment comprising consecutive histidine residues comprises at least 4 consecutive histidine residues.

11. The nucleic acid of claim 10, wherein the peptide segment comprising consecutive histidine residues comprises at least 6 consecutive histidine residues.

12. The nucleic acid of claim 11, wherein the peptide segment comprising consecutive histidine residues comprises at least 8 consecutive histidine residues.

13. The nucleic acid of any one of claims 1-3, wherein the nucleic acid is DNA.

14. The nucleic acid of claim 13, wherein the DNA is cDNA.

15. The nucleic acid of any one of claims 1-3, wherein the nucleic acid is RNA.

16. A replicable vector which comprises the nucleic acid of any one of claims 1-3.

17. The vector of claim 16, wherein the vector is a plasmid, cosmid, λ phage or YAC.

18. A host cell which comprises the vector of claim 16.

19. The cell of claim 18, wherein the cell is a bacterial cell.

20. The bacterial cell of claim 19, wherein the bacterial cell is E. coli.

21. The bacterial cell of claim 20, which comprises a thioredoxin reductase mutation.

22. The bacterial cell of claim 21, which further comprises a glutathione reductase mutation.

23. The cell of claim 18, wherein the cell is a eukaryotic cell.

24. A host-vector system for the production of a soluble polypeptide which comprises the vector of claim 16 and a suitable host cell.

25. A method for producing a soluble polypeptide which comprises growing the host vector system of claim 24 under conditions permitting production of the soluble polypeptide and recovering the soluble polypeptide so produced.

26. A soluble polypeptide encoded by the nucleic acid of any one of claims 1-3.

27. A soluble polypeptide which comprises an extracellular domain of a gonadotropin receptor and thioredoxin, wherein the soluble polypeptide is capable of binding to the gonadotropin.

28. A soluble polypeptide which comprises an extracellular domain of a gonadotropin receptor and a peptide segment comprising consecutive histidine residues, wherein the soluble polypeptide is capable of binding to the gonadotropin.

29. The soluble polypeptide of claim 27, wherein the polypeptide further comprises a peptide segment comprising consecutive histidine residues.

30. The soluble polypeptide of any one of claims 27-29, wherein the gonadotropin receptor is a human luteinizing hormone/choriogonadotropin receptor and the soluble polypeptide is capable of binding to human luteinizing hormone or human chorionic gonadotropin.

31. The soluble polypeptide of any one of claims 27-29, wherein gonadotropin receptor is a human follicle stimulating hormone receptor and the soluble polypeptide is capable of binding to human follicle stimulating hormone.

32. A method of identifying an antibody capable of binding to an extracellular domain of a gonadotropin receptor which comprises:
(a) administering the polypeptide of claim 26 to a subject and obtaining antiserum from the subject;
(b) contacting a gonadotropin receptor with the antiserum;
(c) determining whether any antibody present in the antiserum binds to the a gonadotropin receptor and isolating such antibody,
so as to thereby identify an antibody capable of binding to the extracellular domain of a gonadotropin receptor.

33. A method of obtaining a composition which comprises:
(a) identifying an antibody capable of binding to an extracellular domain of a gonadotropin receptor by the method of claim 32; and
(b) admixing the antibody so identified with a carrier.

34. A method of preventing a subject from becoming pregnant which comprises administering to the subject an amount of an antibody effective to bind to an extracellular domain of a gonadotropin receptor so as to thereby prevent the subject from becoming pregnant.

35. A method of preventing a subject from becoming pregnant which comprises administering to the subject an amount of the polypeptide of claim 26 or an extracellular domain of a gonadotropin receptor so as to thereby prevent a subject from becoming pregnant.

36. A method of terminating a pregnancy in a subject which comprises administering to the subject an amount of an antibody effective to bind to an extracellular domain of a gonadotropin receptor so as to thereby terminate the pregnancy in the subject.

37. A method of terminating a pregnancy in a subject which comprises administering to the subject an amount of the polypeptide of claim 26 or an extracellular domain of a gonadotropin receptor so as to thereby terminate the pregnancy in the subject.

38. A method of stimulating or enhancing production of an antibody capable of binding to an extracellular domain of a gonadotropin receptor in a subject which comprises administering to the subject an amount of the polypeptide of claim 26 or an extracellular domain of a gonadotropin receptor effective to stimulate or enhance antibody production in the subject.

39. A method of treating a cancer in a subject which comprises administering to the subject an amount of the polypeptide of claim 26 or an extracellular domain of a gonadotropin receptor effective to stimulate or enhance production of an antibody capable of binding to an extracellular domain of a gonadotropin receptor so as to thereby treat the cancer in the subject.

40. A method of treating a cancer in a subject which comprises administering to the subject an amount of an antibody effective to bind to an extracellular domain of a gonadotropin receptor so as to thereby treat a cancer in the subject.

41. A. method of preventing a cancer in a subject which comprises administering to the subject an amount of the polypeptide of claim 26 or an extracellular domain of a gonadotropin receptor effective to stimulate or enhance production of an antibody capable of binding to an extracellular domain of a gonadotropin receptor so as to thereby prevent the cancer in the subject.

42. A method of preventing a cancer in a subject which comprises administering to the subject an amount of an antibody effective to bind to an extracellular domain of a gonadotropin receptor so as to thereby prevent a cancer in the subject.

43. The method of any one of claim 39-42, wherein the cancer is lung cancer, bladder cancer, prostate cancer, colorectral cancer, ovarian cancer, cervical cancer, squamous cell cancer, or breast cancer.

44. A method of decreasing a subject's production of androgen which comprises administering to the subject an amount of the polypeptide of claim 26 or an extracellular domain of a gonadotropin receptor effective to stimulate production of an antibody capable of binding to an extracellular domain of a gonadotropin receptor in the subject, so as to thereby decrease the subject's production of androgen.

45. A method of decreasing a subject's production of androgen which comprises administering to the subject an amount of an antibody effective to bind to an extracellular domain of a gonadotropin receptor so as to thereby decrease the subject's production of androgen.

46. A method of preventing a subject from becoming afflicted with ovarian hyperstimulatory syndrome which comprises administering to the subject an amount of an antibody effective to bind to an extracellular domain of a gonadotropin receptor so as to thereby prevent the subject from becoming afflicted with ovarian hyperstimulatory syndrome.

47. A method of treating a subject afflicted with ovarian hyperstimulatory syndrome which comprises administering to the subject an amount of an antibody effective to bind to an extracellular domain of a gonadotropin receptor so as to thereby treat the subject afflicted with ovarian hyperstimulatory syndrome.

48. The method of any one of claims 34-47, wherein the gonadotropin receptor is human luteinizing hormone/choriogonadotropin receptor.

49. The method of any one of claims 34-47, wherein the gonadotropin receptor is follicle stimulating hormone receptor.
